(19) United States
(12) Patent Application Publication
(10) Pub. No.: US 2022/0296841 A1
(43) Pub. Date: Sep. 22, 2022

(54) KNITTED HEADGEAR FOR RESPIRATORY INTERFACE

(71) Applicant: Fisher & Paykel Healthcare Limited, Auckland (NZ)

(72) Inventors: Jonathan Tong Lok SNG, Auckland (NZ); Nigel TRINIDADE, Auckland (NZ); Matthew Roger STEPHENSON, Auckland (NZ); Tony William SPEAR, Auckland (NZ); Jeremy Owen YOUNG, Auckland (NZ)

(21) Appl. No.: 17/753,631

(22) PCT Filed: Sep. 16, 2020
(86) PCT No.: PCT/NZ2020/050104

§ 371 Date: Mar. 9, 2022

Related U.S. Application Data
(60) Provisional application No. 62/901,188, filed on Sep. 16, 2019.

(51) Int. Cl. A61M 16/06

(52) U.S. Cl. CPC A61M 16/0683; A61M 16/0611; A61M 16/0622; A61M 16/0666; A61M 2016/0661

(57) ABSTRACT

A headgear for a respiratory interface on the head of a user includes a first strap, a second strap, and a junction between the ends of the first strap the second strap. The straps are constructed as a tubular knitted structure having a front side configured to be positioned toward the user and a rear side configured to be positioned away from the user. The junction is constructed as a knitted structure having a front side and a rear side. In at least a portion of the junction, the front side and the rear side are joined to one another. The junction can comprise a gusset that spaces the straps apart from one another.

### BACKGROUND

### Field

The present disclosure relates to respiratory interfaces. In particular, the present disclosure relates to headgear for a respiratory interface, such as a headgear having a knitted structure.

### Description of Related Art

Headgear is typically used to secure the patient interface on the user's head. The headgear typically comprises one or more straps and/or panels that are secured to the patient interface and which pass around one or more parts of the patient's head. The headgear is typically configured to provide one or more of following functions: to locate the patient interface in the desired position on the user's face, to maintain the seal between the patient interface and the user's face by application of compression forces between the seal of the patient interface and the user's face, to distribute the required forces between the patient interface and the user, to improve the comfort of the patient during therapy, to improve the appearance of the product, or to be cost effective to manufacture. Such headgear can comprise a number of different sizes and configurations of straps that are integrally formed, or joined together. Further achieving one or more of the above functions can be difficult, as one function can conflict with another. Thus, a need exists for headgear arrangements that improve upon one or more of the above functions or at least provide the public with a useful choice.

### SUMMARY

The systems, methods and devices described herein have innovative aspects, no single one of which is indispensable or solely responsible for their desirable attributes. Without limiting the scope of the claims, some of the advantageous features will now be summarized.

An aspect of the present disclosure involves a headgear for supporting a respiratory interface on the head of a user. The headgear includes a first strap having a first end and a second end. The headgear includes a second strap having a first end and a second end. The headgear includes a first junction between the first end of the first strap and the first end of the second strap and a second junction between the second end of the first strap and the second end of the second strap. Each of the first strap and the second strap is constructed as a tubular knitted structure having a front side configured to be positioned toward the user and a rear side configured to be positioned away from the user. Each of the first junction and the second junction is constructed as a knitted structure having a front side configured to be positioned toward the user and a rear side configured to be positioned away from the user. The front side and the rear side are joined to one another in at least a portion of each of the first junction and the second junction.

In some configurations, the front side and the rear side are joined to one another in only a portion of each of the first junction and the second junction.

In some configurations, the front side and the rear side are joined to one another in an outer portion of each of the first junction and the second junction.

In some configurations, the front side and the rear side are joined to one another in an entirety of each of the first junction and the second junction.

In some configurations, the first junction comprises a first outer portion, a second outer portion and a first gusset portion. The first outer portion is located adjacent the first end of the first strap. The second outer portion is located adjacent the first end of the second strap. The first gusset portion extends between and separates the respective first ends of the first and second straps. The second junction comprises a first outer portion, a second outer portion and a second gusset portion. The first outer portion is located adjacent the second end of the first strap. The second outer portion is located adjacent the second end of the second strap. The second gusset portion extends between and separates the respective second ends of the first and second straps.

In some configurations, the front side and the rear side are joined to one another within an entirety of each of the first and second gussets.

In some configurations, one or both of the first strap and the second strap comprise one or more relatively inelastic yarns and one or more relatively elastic yarns.

In some configurations, the front side of one or both of the first and the second straps has smaller loop length than the rear side to create curvature along a length of the strap.

In some configurations, one or both of the first junction and the second junction comprise one or more relatively inelastic yarns and one or more relatively elastic yarns.

In some configurations, the front side of one or both of the first and the second junctions has smaller loop length than the rear side to create curvature along a length of the junction.

In some configurations, the first and second straps and the first and second junctions form a rear portion of the headgear, wherein the first strap is an upper strap and the second strap is a lower strap.

In some configurations, each of the first and second junctions are configured for connection to a front strap or front straps of the headgear.

In some configurations, a first overmolded connector is attached to the first junction and a second overmolded connector is attached to the second junction, each of the first and second overmolded connectors comprising an attachment location for the front strap or front straps.

In some configurations, an area of the joining between the front side and the rear side is greater than an area of the first or second junction covered by the connector.

In some configurations, an area of the joining between the front side and the rear side is less than an area of the first or second junction covered by the connector.

In some configurations, an area of the joining between the front side and the rear side is equal to or substantially equal to an area of the first or second junction covered by the connector.

In some configurations, each of the first junction and the second junction comprises an open-work structure that defines an attachment opening for the front strap or front straps.

In some configurations, the headgear includes the front strap or front straps, wherein the front strap or front straps are less elastic than at least the upper strap and the lower strap of the rear portion.

In some configurations, a length of the first strap is greater than a length of the second strap.

In some configurations, a length of the second strap is greater than a length of the first strap.

In some configurations, a length of the first strap is equal to or substantially equal to a length of the second strap.

In some configurations, the joining of the front side and the rear side within each of the first junction and the second junction comprises interlocking of the front side and the rear side.

In some configurations, the joining of the front side and the rear side within the first junction and the second junction comprises one or more of stitching, thermoforming, gluing, overmolding, welding or a joint created by introducing molten plastic between the front side and the rear side and allowing the molten plastic to cool and solidify.

In some configurations, the front sides of the first strap and the second strap merge to form the front sides of one or both of the first junction and the second junction.

In some configurations, the rear sides of the first strap and the second strap merge to form the rear sides of one or both of the first junction and the second junction.

In some configurations, a thickness of one or both of the first junction and the second junction is equal to or less than a thickness of one or the other of the first strap and the second strap.

An aspect of the present disclosure involves a headgear for supporting a respiratory interface on the head of a user. The headgear includes a knitted body portion having a first strap having a first end and a second end, and an inner edge and an outer edge. The knitted body portion has a second strap having a first end and a second end, and an inner edge and an outer edge. The inner edge of the first strap faces the inner edge of the second strap. A first gusset extends between and connects the first end of the first strap and the first end of the second strap. The first gusset has an inner edge. A second gusset extends between and connects the second end of the first strap and the second end of the second strap. The second gusset has an inner edge. Each of the first gusset and the second gusset is configured to space apart the first strap and the second strap such that the respective inner edges of the first gusset and the second gusset extend between the respective inner edges of the first strap and the second strap.

In some configurations, the first strap has a first strap width and the second strap has a second strap width.

In some configurations, the first strap width is equal or approximately equal to the second strap width.

In some configurations, each of the first strap width and the second strap width is between about 5-15 mm.

In some configurations, each of the first strap width and the second strap width is 10 mm or about 10 mm.

In some configurations, the inner edge of the first gusset defines a first gusset width and the inner edge of the second gusset defines a second gusset width.

In some configurations, each of the first gusset width and the second gusset width is greater than the first strap width and/or the second strap width.

In some configurations, each of the first gusset width and the second gusset width is at least twice, at least three times, or at least five times as large as the first strap width and/or the second strap width.

In some configurations, each of the first gusset width and the second gusset width is between about 15 mm-45 mm, between about 20 mm-40 mm, or between about 25 mm-35 mm.

In some configurations, each of the first gusset width and the second gusset width is about 30 mm.

In some configurations, each of the first gusset and the second gusset has a first side and a second side, wherein the first side and the second side of each of the first and second gussets defines a gusset angle therebetween of between about 30-90 degrees with the knitted body portion laid flat in a resting position.

In some configurations, the gusset angle of each of the first and second gussets is about 70 degrees.

In some configurations, the knitted body and the first and second gussets are configured such that the first and second straps can be oriented to form an angle of between about 30-180 degrees in response to an application of force tending to separate the first and second straps.

In some configurations, a maximum distance between the inner edges of the first and second straps is between about 20 mm-150 mm, between about 25 mm-100 mm, between about 30 mm-50 mm with the knitted body portion laid flat in the resting position.

In some configurations, a maximum distance between the inner edges of the first and second straps is 38 mm or about 38 mm with the knitted body portion laid flat in the resting position.

In some configurations, a distance between the inner edges of the first and second gussets is between about 150 mm-250 mm, between about 175 mm-225 mm, or between about 190 mm-200 mm, about 200 mm with the knitted body portion laid flat in the resting position.

In some configurations, a distance between the inner edges of the first and second gussets is 195 mm or about 195 mm with the knitted body portion laid flat in the resting position.

In some configurations, the knitted body is configured such that the distance between the inner edges of the first and second gussets is between about 250 mm-375 mm, between about 275 mm-350 mm, or between about 300 mm-325 mm when a force of 10 Newtons is applied in a direction aligned with or parallel to the distance.

In some configurations, the knitted body is configured such that the distance between the inner edges of the first and second gussets is 315 mm or about 315 mm when a force of 10 Newtons is applied in a direction aligned with or parallel to the distance.

In some configurations, a distance between an outer end and the inner edge of each of the first and second gussets is between about 15 mm-45 mm, between about 20 mm-40 mm, or between about 25 mm-35 mm.

In some configurations, a distance between an outer end and the inner edge of each of the first and second gussets is 30 mm or about 30 mm.

In some configurations, the inner edges of one or both of the first gusset and the second gusset comprise a binding.

In some configurations, the outer edges of one or both of the first gusset and the second gusset comprise a binding.

An aspect of the present disclosure involves a headgear for supporting a respiratory interface on the head of a user. The headgear includes a first strap having an end, a second strap having an end, and a junction between the end of the first strap and the end of the second strap. Each of the first strap and the second strap is constructed as a tubular knitted structure having a front side configured to be positioned toward the user and a rear side configured to be positioned away from the user. At least a portion of the junction is constructed as a knitted structure having a front side configured to be positioned toward the user and a rear side configured to be positioned away from the user. The front side and the rear side are joined to one another.

In some configurations, the front side and the rear side are joined to one another in only a portion of the junction.

In some configurations, the front side and the rear side are joined to one another in an outer portion of the junction.

In some configurations, the front side and the rear side are joined to one another in an entirety of the junction.

In some configurations, the junction comprises a first outer portion, a second outer portion and a gusset portion. The first outer portion is located adjacent the end of the first strap. The second outer portion is located adjacent the end of the second strap. The gusset portion extends between and separates the respective ends of the first and second straps.

In some configurations, the front side and the rear side are joined to one another within an entirety of the gusset.

In some configurations, one or both of the first strap and the second strap comprise one or more relatively inelastic yarns and one or more relatively elastic yarns.

In some configurations, the front side of one or both of the first and the second straps has smaller loop length than the rear side to create curvature along a length of the strap.

In some configurations, the junction comprises one or more relatively inelastic yarns and one or more relatively elastic yarns.

In some configurations, the front side of the junction has smaller loop length than the rear side to create curvature along a length of the junction.

In some configurations, the first and second straps and the junction form a rear portion of the headgear, wherein the first strap is an upper strap and the second strap is a lower strap.

In some configurations, the junction is configured for connection to a front strap of the headgear.

In some configurations, the headgear includes an overmolded connector attached to the junction. The connector includes an attachment location for the front strap.

In some configurations, an area of the joining between the front side and the rear side is greater than an area of the junction covered by the connector.

In some configurations, an area of the joining between the front side and the rear side is less than an area of the junction covered by the connector.

In some configurations, an area of the joining between the front side and the rear side is equal to or substantially equal to an area of the junction covered by the connector.

In some configurations, the junction comprises an open-work structure that defines an attachment opening for the front strap.

In some configurations, the headgear includes the front strap. The front strap is less elastic than at least the upper strap and the lower strap of the rear portion.

In some configurations, a length of the first strap is greater than a length of the second strap.

In some configurations, a length of the second strap is greater than a length of the first strap.

In some configurations, a length of the first strap is equal to or substantially equal to a length of the second strap.

In some configurations, the joining of the front side and the rear side within the junction comprises interlocking of the front side and the rear side.

In some configurations, the joining of the front side and the rear side within the junction comprises one or more of stitching, thermoforming, gluing, overmolding, welding or a joint created by introducing molten plastic between the front side and the rear side and allowing the molten plastic to cool and solidify.

In some configurations, the front sides of the first strap and the second strap merge to form the front side of the junction.

In some configurations, the rear sides of the first strap and the second strap merge to form the rear side of the junction.

In some configurations, a thickness of the junction is equal to or less than a thickness of one or the other of the first strap and the second strap.

An aspect of the present disclosure involves a method of making a headgear for a respiratory interface. The method includes knitting a first strap to have a tubular structure. The method includes knitting a second strap to have a tubular structure. The method includes knitting a junction to couple the first strap to the second strap. The method includes joining a front side and a rear side of at least a portion of the junction.

In some configurations, the knitting of the junction is done prior to the knitting of the first strap and the second strap.

In some configurations, the knitting of the junction is done after the knitting of the first strap and the second strap.

In some configurations, the junction and the first strap are knitted via a first yarn carrier and the second strap is knitted via a second yarn carrier.

In some configurations, the knitting of at least the first strap and the second strap comprising knitting with both a relatively inelastic yarn and a relatively elastic yarn.

In some configurations, the knitting of the junction comprises knitting a gusset configured to space apart the first strap portion and the second strap portion.

In some configurations, the method includes binding off an inner edge of the gusset.

In some configurations, the method includes binding on an outer edge of the junction.

In some configurations, the method includes binding on an inner edge of the gusset.

In some configurations, the method includes binding off an outer edge of the junction.

In some configurations, the outer edge has a smaller length than the inner edge.

In some configurations, the length of the outer edge is between one-tenth and one-half, or one-fifth and one-third, or about one-third of the length of the inner edge.

In some configurations, the joining of the front side and the rear side of the junction comprises crossing over one or more yarns between the front side and the rear side to interlock the front side and the rear side.

In some configurations, the joining of the front side and the rear side of the junction is done during the knitting of the junction.

An aspect of the present disclosure involves a headgear for supporting a respiratory interface on the head of a user. The headgear includes a knitted body portion having a first strap having an inner edge and an outer edge. The knitted body portion has a second strap having an inner edge and an outer edge. The inner edge of the first strap faces the inner edge of the second strap. A gusset extends between and connects an end of the first strap and an end of the second strap. The gusset has an inner edge comprising at least one binding stitch. The gusset is configured to space apart the first strap and the second strap such that the inner edge of the gusset extends between the respective inner edges of the first strap and the second strap.

In some configurations, the at least one binding stitch is configured to bind-off loops of the gusset.

In some configurations, the at least one binding stitch is configured to bind-on loops of the gusset.

In some configurations, the gusset further comprises an outer edge comprising at least one binding stitch.

In some configurations, the at least one binding stitch of the outer edge is configured to bind-off loops of the gusset.

In some configurations, the at least one binding stitch of the outer edge is configured to bind-on loops of the gusset.

An aspect of the present disclosure involves a headgear for supporting a respiratory interface on the head of a user. The headgear includes a knitted body portion with a first tubular knitted strap having a first front side and a first rear side joined at an inner edge and an outer edge. The knitted body portion includes a second tubular knitted strap having a second front side and a second rear side joined at an inner edge and an outer edge. The inner edge of the first strap faces the inner edge of the second strap. A knitted gusset extends between and connects an end of the first tubular knitted strap and an end of the second tubular knitted strap. The gusset includes a first gusset side, a second gusset side, and an inner edge comprising at least one binding stitch securing the first gusset side to the second gusset side. The gusset is configured to space apart the first strap and the second strap in a resting position such that the inner edge of the gusset extends between the respective inner edges of the first strap and the second strap.

In some configurations, the at least one binding stitch is configured to bind-off loops of the gusset.

In some configurations, the at least one binding stitch is configured to bind-on loops of the gusset.

In some configurations, the gusset further comprises an outer edge comprising at least one binding stitch.

In some configurations, the at least one binding stitch of the outer edge is configured to bind-off loops of the gusset.

In some configurations, the at least one binding stitch of the outer edge is configured to bind-on loops of the gusset.

An aspect of the present disclosure involves a bracket for connecting headgear straps. A further aspect of the present disclosure involves a headgear comprising straps connected by a bracket.

In some configuration, the bracket comprises a first bracket portion comprising a first inner surface and a second bracket portion comprising a second inner surface. At least one hinge connects the first bracket portion to the second bracket portion. The hinge is configured to allow the first bracket portion and the second bracket portion to be moved from a first open position to a second closed position. In the second closed position, the first inner surface and the second inner surface face each other.

In some configurations, the bracket comprises an elongate slot adjacent the hinge.

In some configurations, the bracket further comprises an upper connector configured to connect to a top strap and a lower connector configured to connect to a rear strap.

In some configurations, the upper connector is formed by the first bracket portion and the second bracket portion.

In some configurations, at least the lower connector is formed by the first bracket portion and the second bracket portion.

In some configurations, the elongate slot comprises portions formed in the first bracket portion and the second bracket portion.

In some configurations, the elongate slot is configured to receive a front strap.

In some configurations, the hinge is positioned adjacent a periphery of the first bracket portion and the second bracket portion.

In some configurations, the hinge comprises a hinge axis that is parallel to an axis of the elongate slot.

In some configurations, recesses are provided on the first inner surface.

In some configurations, the recesses form a pocket in the closed position.

In some configurations, energy directors are provided on the second inner surface and the first bracket portion and the second bracket portion are welded together via the energy directors.

In some configurations, in the second closed position, the recess forms at least one pocket in one of the upper connector and the lower connector.

In some configurations, at least one strap or at least one strap end is sandwiched between the first inner surface and the second inner surface.

In some configurations, at least one strap is secured within the at least one pocket.

In some configurations, an end of the at least one strap is secured to the first and second inner surfaces by adhesive.

In some configurations, the first bracket portion and the second bracket portion are secured in the closed position by a snap fit.

In some configurations, the bracket is symmetrical about a minor axis of the elongate slot.

In some configurations, the first bracket portion is a mirror of the second bracket portion.

In some configurations, the first bracket portion, the second bracket portion, and the hinge are integrally formed.

In some configurations, the first bracket portion, the second bracket portion, and the hinge are integrally moulded.

In some configurations, one of the first bracket portion or the second bracket portion is a flap. In some configurations, the flap comprises the inner surface.

An additional aspect of the present disclosure involves a bracket for connecting headgear straps the bracket comprising an arm that extends from a front connector, the front connector comprising an elongate slot. A further aspect of the present disclosure involves a headgear comprising straps connected by the bracket.

In some configurations, the bracket comprises flaps connected to the front connector by a hinge.

In some configurations, the hinge comprises a hinge axis that is angled with respect to an axis of the elongate slot.

In some configurations, a headgear comprises the bracket, and the bracket comprises an arm extending from a position adjacent the hinge.

In some configurations, a headgear comprises the bracket and at least one strap connected to the arm.

In some configurations, in the closed position, the inner surface is adjacent the arm, the at least one strap being sandwiched between the inner surface and the arm.

In some configurations, the at least one strap comprises a hollow strap end, the arm being received within the hollow strap end.

In some configurations, the hinge axis is perpendicular to the at least one strap.

In some configurations, the hinge axis is perpendicular to the arm.

In some configurations, the bracket comprises a second hinge and an adjacent flap positioned on the opposite side of the arm.

In some configurations, the hinges are adjacent the elongate slot of the front connector.

In some configurations, the arm is received inside an end of at least one strap.

In some configurations, the hinges allow the flaps to clamp over an end of at least one strap.

In some configurations a strap is sandwiched between the flaps and the arm.

In some configurations, the flaps are welded in the closed position.

In some configurations, the arms extend from the front connector non-parallel to each other.

In some configurations, the bracket comprises a symmetrical arrangement.

In some configurations, the bracket comprises an upper connector and a lower connector.

In some configurations the upper connector and the lower connector comprise different configurations, such as different lengths or widths, attachment mechanisms, sizes, angles, or removability.

A further aspect of the present disclosure involves a mask frame for a respiratory interface. The mask frame includes a mask frame inlet and a mask frame outlet. The mask frame includes at least one lateral frame portion comprising a front surface and a rear surface, with a headgear connector extending from the front surface. An opening is defined through the at least one lateral frame portion, from the front surface to the rear surface, and positioned between the headgear connector and the mask frame inlet.

In some configurations, the at least one headgear connector comprises a post positioned adjacent to the opening.

In some configurations, the headgear connector comprises a medial flange extending medially from the post.

In some configurations, a medial flange extends above the opening.

In some configurations, the mask frame comprises a gap between the medial flange and the front surface of the at least one lateral frame portion.

In some configurations, the mask frame comprises a lateral flange extending laterally from the post.

In some configurations, the mask frame comprises an upper surface of the post is continuous with an upper surface of the medial flange and an upper surface of the lateral flange.

In some configurations, the mask frame comprises a lower surface of the post is continuous with a lower surface of the medial flange and a lower surface of the lateral flange.

In some configurations, a width of the at least one lateral frame portion is greater than a width of the post.

In some configurations, the width of the post is substantially the same as a width of the medial flange.

In some configurations, the width of the post is substantially the same as a width of the lateral flange.

In some configurations, a length of the lateral flange is greater than a length of the medial flange.

In some configurations, the length of the lateral flange is approximately 4.2 mm and the length of the medial flange is approximately 0.5 mm.

In some configurations, a thickness of the lateral flange is greater than a thickness of the medial flange.

In some configurations, the thickness of the lateral flange is approximately 1.5 mm and the thickness of the medial flange is approximately 1 mm.

In some configurations, the lateral flange extends beyond a lateral edge of the side arm.

In some configurations, a front surface of the post is continuous with a front surface of the medial flange and a front surface of the lateral flange.

In some configurations, the front surface of the lateral flange has a curvature following the curvature of the user's face.

In some configurations, the post has a rectangular shape or cross-section.

In some configurations, the post has a length of 6.5 mm and a width of 3.9 mm.

In some configurations, the mask frame comprises a recess positioned between the opening and the mask frame inlet.

In some configurations, the recess is positioned on the rear surface of the side arm.

A further aspect of the present disclosure involves a headgear strap configured to mate with the above referenced headgear connector, wherein the headgear strap has an opening configured to secure to the headgear connector.

In some configurations, the opening is located on the free end of the headgear strap.

In some configurations, the opening has a rectangular shape corresponding with the shape of the post.

In some configurations, when assembled, the thickness of the strap fits within the gap.

In some configurations, when assembled, the medial flange and lateral flange hang over the strap.

In some configurations, the headgear strap is formed of a flexible material.

In some configurations, the opening is formed through removal of material such as die-cutting and RF crease-cutting.

In some configurations, the free end of the strap comprises an overmoulded section formed with a resilient material.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through the use of the accompanying drawings.

FIG. 1 is a schematic representation of a respiratory system configured to supply pressurized and humidified breathing gases to a user through a patient interface.

FIG. 2 is a side view of a patient interface assembly positioned on a user. The patient interface includes an interface in the form of a mask and a headgear arrangement.

FIG. 3 is a plan view of a rear portion of the headgear arrangement of FIG. 2.

FIG. 4 is another plan view of the rear portion of the headgear arrangement of FIG. 2.

FIG. 5 is a plan view of an embodiment of the rear portion in which outer portions of junctions between the headgear straps are joined.

FIG. 6 is a plan view of an embodiment of the rear portion in which entireties of junctions between the headgear straps are joined.

FIG. 7 is a plan view of an embodiment of the rear portion in which gusset portions of junctions between the headgear straps and tips of the headgear straps are joined.

FIG. 8 is a plan view of an embodiment of the rear portion in which gusset portions of junctions between the headgear straps are joined.

FIG. 9 is a section view of a tubular portion of the rear portion of the headgear.

FIG. 10 is a section view of a joined portion of the rear portion of the headgear.

FIG. 11 is a plan view of the rear portion of the headgear illustrating a knitting direction and process.

FIG. 12 is a schematic view of the rear portion of the headgear illustrating portions formed by different yarn carriers.

FIG. 13 illustrates different loop lengths between a front side and a rear side of the rear portion of the headgear.

FIGS. 14A and 14B illustrate the effect of different loop lengths to provide curvature to the rear portion of the headgear.

FIGS. 15A and 15B illustrate an example of plating of two types of yarn in the rear portion of the headgear.

FIG. 16 illustrates a process of making the rear portion of the headgear.

FIG. 17 is a perspective view of a headgear assembly including a bracket.

FIG. 18 is a plan view of a bracket for use in the headgear assembly, according to an embodiment of the present disclosure.

FIGS. 19, 20 and 21 show the bracket in an open position.

FIG. 22 shows a cross section of the bracket and a strap in the closed position.

FIG. 23 shows the bracket in a closed position, along with the upper strap and the lower strap.

FIG. 24 is a perspective view a bracket for the headgear assembly according to another embodiment of the present disclosure.

FIG. 25 shows the bracket in an open position.

FIGS. 26 and 27 provide additional views of the bracket in a closed position.

FIG. 28 shows the bracket in a closed position with straps attached to the bracket.

FIGS. 29, 30, and 31 show a bracket according to another embodiment of the present disclosure.

FIGS. 32, 33, 34, and 35 are front, rear, top, and bottom views of a mask frame.

FIGS. 36, 37, and 38 provide additional views of the mask frame.

### DETAILED DESCRIPTION

Embodiments of systems, components and methods of assembly and manufacture will now be described with reference to the accompanying Figures, wherein like numerals refer to like or similar elements throughout. Although several embodiments, examples and illustrations are disclosed below, it will be understood by those of ordinary skill in the art that the inventions described herein extends beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

FIG. 1 illustrates an example respiratory therapy system suitable for supplying breathing gases to a user for positive airway pressure (PAP) therapy (e.g., continuous positive airway pressure (CPAP) therapy) or non-invasive ventilation (MV) therapy. The example respiratory therapy system 100 may include a gas source 102, a humidifier 104, a patient interface 150 and a breathing gas circuit 106 that connects the humidifier 104 (or gas source 102) to the patient interface 150. The gas source 102 can provide a supply of breathing gas to the humidifier 104. The gas source 102 may comprise a blower in which breathing gas, e.g., ambient air, is drawn into the gas source 102 through an inlet 110 in the gas source casing by an impeller 112. The rotational speed of the impeller 112 may be modulated to regulate the quantity of air drawn into the gas source 102 and the supply of breathing gas delivered to the respiratory therapy system 100. Breathing gas may include any single gas or multiple gases that are breathable by a user of the system 100.

The pressure and/or flow rate of breathing gas exiting the gas source 102 may be regulated by a controller 114. The controller 114 may modulate the rotational speed of the impeller 112 according to one or more predetermined algorithms and in accordance with one or more user inputs that may be provided via a user input 116.

The gas source 102 represents an actively controlled flow generator. Other gas sources, such as a compressed air cylinder with suitable pressure or flow regulation, may also be used to supply breathing gas. The outlet of the gas source 102 may be coupled to a separate humidifier 104. The humidifier 104 may be configured to heat and/or humidify the breathing gas prior to delivery, e.g., delivery to the user. In some embodiments, the humidifier is integrated with the gas supply. The humidifier 104 may include a base 120 and a humidifier chamber 122. The chamber 122 may be configured to hold humidification fluid 124, such as water, and may be disengaged, e.g., temporarily disengaged or permanently disengaged, from the humidifier base 120 to allow it to be filled or replaced. The humidifier 104 receives gases from the gas source 102 through chamber inlet 126. The humidifier base 120 can include a heater such as a heater plate 130. The chamber 122 rests on the heater plate 130 when engaged with the humidifier base 120. The heater plate 130 dissipates heat, e.g., heat generated by electrical resistance, to the chamber 122. The chamber 122 preferably has a heat conductive base to enable the heat generated by the heater plate 130 to pass efficiently to the humidification fluid 124. Controller 114 can also control the humidifier 104, and in particular the supply of electrical energy to the heater plate 130, to regulate any function of the humidifier 104, e.g., the temperature and humidity of the breathing gas supplied to the user.

The breathing gas can be supplied to the user via a chamber outlet 132 and the breathing gas circuit 106 in the form of a conduit which may incorporate a heating or warming element, e.g., a heater wire, to heat or warm (e.g., keep hot or warm) the breathing gases during transportation to the patient interface 150. The electrical energy supplied to the heater wire may be controlled by the controller 114. The controller 114 may receive feedback from one or more sensors incorporated in a control network throughout the respiratory therapy system to monitor properties of the breathing gas, such as, but not limited to, pressure, flow, temperature, and/or humidity.

The patient interface 150 couples the user with the respiratory therapy system 100, such that gases, e.g., heated and humidified gases from the humidifier 104, may be delivered to the user's respiratory system. Breathing gases can be delivered to the user at, or near, optimal temperature and humidity (e.g., warmed and fully saturated with water vapor at temperatures of between 27 and 37° C.) as the gases are delivered to the user's nares and or mouth. Emulating the conditions within healthy adult lungs (37° C., 44 mg/L humidity) can help maintain healthy mucocilliary function in users with respiratory disorders affecting secretion and for all patients humidifying the gas helps maintain comfort and compliance. A number of different styles of patient interface 150, such as those disclosed herein, may be used in the example system 100 or a similar system.

FIG. 2 illustrates an example patient interface assembly 150 that can be used in the system 100 of FIG. 1. The interface assembly 150 generally includes an interface (e.g., a mask) 152 and a headgear arrangement (“headgear”) 154. The interface 152 is configured to deliver the flow of breathing gases to an airway of the user. The headgear 154 is configured to secure the interface 152 in place on the user.

The illustrated interface 152 is a mask, which can define a breathing chamber. In particular, the illustrated interface 152 is a nasal mask. The illustrated nasal mask 152 is a direct nasal mask having nasal elements (e.g., nasal pillows) configured to deliver the flow of breathing gases directly to the nares of the user. However, other suitable interfaces could also be used with the headgear 154 and/or the headgear 154 could be modified for use with different types of interfaces. For example, the interface could be an indirect nasal mask that creates a seal on the user's face surrounding one or both nares or a full face mask that creates a seal on the user's face surrounding the mouth and nares. The patient interface 150 can include a frame and a cushion or seal. The cushion or seal can be directly coupled to the frame or can form a portion of a cushion module that is connectable to the frame. Such a cushion module can include a relatively rigid housing or clip portion that is constructed from a material(s) that is harder than the material of the cushion. The relatively rigid housing or clip portion can interface with the frame.

The cushion or seal can be constructed from a material(s) that is softer than the material of the relatively rigid housing or clip portion, such as silicone or a similar material. Unless indicated otherwise, descriptions of relative rigidity or stiffness of materials herein refer to differences in the modulus of elasticity between the materials or differences in rigidity or stiffness of solid bodies of the same size and shape constructed from the materials being compared. Descriptions of relative rigidity or stiffness of particular structures can refer to resistance to deformation of those structures, taking into account both material properties and physical characteristics of the structures (e.g., size, shape, wall thickness). The cushion or seal can be an inflation-type seal having an interior-concave curved wall that inflates when the interior of the interface 150 is pressurized to provide a good seal and better conform to the face of the particular user. However, other types of seals can be used, if desired. Moreover, certain features, aspects and advantages of the present disclosure can be utilized with other types of patient interfaces.

The headgear 154 can be coupled to the mask 152 in any suitable manner. For example, the headgear 154 can be coupled to the frame of the mask 152. The frame or mask 152 can include any suitable number or type of headgear mounts. In some configurations, the frame or mask 152 includes two, three, four, five or more headgear mount locations configured to receive two, three, four, five or more straps of a headgear or headgear assembly. However, in the illustrated arrangement, a single headgear strap is provided on each side of the user's head. The single straps on each side can be separate from one another or can form a single strap that extends from one side of the user's head to the other side and has an intermediate portion that connects to the frame or mask 152.

In the illustrated arrangement, the headgear 154 has a front portion 160 and a rear portion 162. The front portion 160 is configured to connect to the mask 152 and extends between the mask 152 and the rear portion 162. The rear portion 162 is configured to engage a side and/or rearward portion of the user's head. As described above, the illustrated front portion 160 includes a single front strap 164 on each side of the interface assembly 150 or head of the user. The front straps 164 on each side of the interface assembly 150 can be separate from one another and separately connected to the mask 152 or can be formed by a single strap 164. References to the front strap 164 herein include both arrangements unless explicitly indicated otherwise.

The illustrated rear portion 162 of the headgear 154 is of a bifurcated arrangement comprising multiple straps. In particular, the rear portion 162 of the headgear 154 includes a first or upper strap 170 and a second, lower or rear strap 172. The first strap 170 and the second strap 172 are configured to be spaced apart from one another on the user's head in use and/or in a resting position, which is described further below. The first strap 170 can be configured to extend over the top of the user's head and the second strap 172 can be configured to extend around the back of the user's head.

In the illustrated arrangement, a gusset 174 is positioned between the ends of the first strap 170 and the second strap 172 on each side of the rear portion 162 of the headgear 154. The gussets 174 separate the straps 170, 172 or facilitates separation of the straps 170, 172 by inhibiting or preventing the straps 170, 172 or at least a portion thereof from being placed directly adjacent one another in use and/or in the resting position. Separation between the straps 170, 172 facilitates correct fitting of the headgear 154 and, thus, the interface assembly 100 and helps the headgear 154 keep its shape and maintain its position on the user's head during use. Each of the gussets 174 along with the portions of the straps 170, 172 positioned alongside the gusset 174 forms a junction 180 between the straps 170, 172.

The rear portion 162 of the headgear 154 can be partially or fully formed by a knitted structure. Thus, the rear portion 162 can form or be referred to herein as a knitted body portion. In some configurations, at least the straps 170, 172 and the gussets 174 are partially or fully formed by a knitted structure. As described further below, the knitted rear portion 162 can include non-knitted structures, such as portions or components configured to connect to the front portion 160 of the headgear 154. In some configurations, the front portion 160 of the headgear 154 is less elastic than the rear portion 162. The front portion 160 can be constructed of a non-knitted material, such as a composite fabric material (e.g., laminated microfiber fabric and UBL material).

Each of the front straps 164 can have a first end connected to the mask 152 and a second end that loops through an opening in the rear portion 162 of the headgear 154. For example, the first end can be passed through an opening in the frame of the mask 152 to create a loop and sewn or otherwise secured to itself. The second end can be connected to an intermediate portion of the strap 164 at a desired location, such as via a hook-and-loop fastener, to adjust a length of the front strap 164. Other suitable arrangements for connecting the first and second ends of the front straps 164 to the mask 152 and the rear portion 162 of the headgear 154 can also be used, such as quick-disconnect connectors, for example.

FIGS. 3 and 4 illustrate an example of the rear portion 162 of the headgear 154 separate from the front portion 160 (e.g., front strap(s) 164) and mask 154. In FIGS. 3 and 4, the rear portion 162 is shown in a resting position. As used herein, the resting position refers to the rear portion 162 (or another specified portion) of the headgear 154 as laid on a flat surface with no external force present. The rear portion 162 may be flat or may have portions that are elevated from the surface as a result of internal biasing forces within the rear portion 162 that provide a non-flat shape to the rear portion 162 (such as variations in loop length, as described further herein).

As described above, the rear portion 162 is partially or wholly constructed by a knitted structure. In some configurations, the knitted structure can be formed as a single piece. In alternative arrangements, the knitted structure can be formed by several discrete knitted pieces that are sewn or otherwise connected to one another. The resulting rear portion 162, regardless of the particular construction, can be considered to have several portions as described above, such as the first strap 170, the second strap 172 and the gussets 174. As described above, end portions of the first strap 170 and the second strap 172 cooperate with the gussets 174 to form the junctions 180 between the straps 170, 172.

The first strap 170 can have a first end and a second end, each identified by the reference number 190 for convenience, and an intermediate portion 192. Similarly, the second strap 172 can have a first end and a second end, each identified by the reference number 194 for convenience, and an intermediate portion 196. The ends 190, 194 of the first and second straps 170, 172, along with the gussets 174, form the junctions 180. However, the ends 190, 194 of the straps 170, 172 may extend inwardly beyond the gusset 174 and/or junction 180. Thus, when the rear portion 162 of the headgear 154 is discussed in the context of the straps 170, 172 and the junctions 180, the “ends” of the straps 170, 172 may be in reference to the portions of the ends 190, 194 adjacent to but outside of (inward of) the junctions 180 or the gussets 174. In other contexts, such as when discussing the junctions 180 themselves, the “ends” of the strap 170, 172 may include portions of the ends 190, 194 within the junctions 180. For convenience, as described herein, each of the junctions 180 has a first outer portion 200 defined by the end 190 of the first strap 170 and a second outer portion 202 defined by the end 194 of the second strap 172. Depending on the particular manufacturing process employed, the outer portions 200, 202 or ends 190, 194 of the straps 170, 172 within the junctions 180 may be readily distinguishable from the gussets 174. If not readily distinguishable, the outer portions 200, 202 may be defined by projections of the straps 170, 172 within the junctions 180. In some configurations, the knitted structure of the straps 170, 172 merge to form the junctions 180. In such configurations, the straps 170, 172 may not be readily distinguishable from the gussets 174.

As described above, each of the gussets 174 extends between and separates the respective ends 190, 194 of the first and second straps 170, 172. Each gusset 174 has an inner end or edge 210 and an outer end or edge 212. The inner ends or edges 210 of the gussets 174 face one another. Each gusset 174 also has an upper edge 214 and a lower edge 216. Each of the straps 170, 172 has an inner edge 220 and an outer edge 222. The inner edges 220 of the straps 170, 172 face one another. The outer edges 222 of the straps 170, 172 face away from one another and, in the illustrated arrangement, define outer edges or upper and lower edges of the rear portion 162 of the headgear 154. The inner edges 210 of the gussets 174 and the inner edges 220 of the straps 170, 172 are spaced apart from one another to define an interior space of the rear portion 162.

In some configurations, at least a portion of the knitted structure of the rear portion 162 of the headgear 154 is a tubular knitted structure. In other words, when viewed in cross-section, the rear portion 162 or portions thereof (e.g., the first strap 170, the second strap 172, the gussets 174 or the junctions 180) have a closed loop structure comprising a first or front side 230 and a second or rear side 232 (FIGS. 9 and 10). The front side 230 can face the user in use and the rear side 232 can face away from the user in use. In some configurations, the front side 230 and rear side 232 are formed as a tubular structure during the knitting process. A tubular structure, and in particular a knitted tubular structure, provides an advantageous amount of stretch and flexibility relative to a single wall or non-tubular structure so as to be able to fit a greater number of patients. In addition, the naturally rounded edge of a tubular strap decreases localized pressure on the head of the user. Furthermore, the tubular structure can allow the straps to hold their shape better than a single wall, non-tubular structure. In other configurations, the rear portion 162 can be knitted (or otherwise manufactured) as a flat, single-walled structure and subsequently formed into a closed loop structure. Ends of the formed structure can be connected by sewing, or another suitable arrangement, to retain the tubular or closed loop structure.

In a tubular structure, the front side 230 and the rear side 232 of the tubular structure are connected along a pair of opposed edges, such as the inner and outer edges 220, 222. The front and rear sides 230, 232 are not connected between the edges 220, 222. In such an arrangement, the front and rear sides 230, 232 remain able to move relative to one another. In other words, the front side 230 and the rear side 232 may be laid against one another, separated to create or maintain a gap therebetween, or shift such that the front side 230 is offset from the rear side 232 (or the locations of the edges 220, 222 and the sides 230, 232 move within the tubular structure). As used herein, the front side 230 and the rear side 232 being able to move means that the front side 230 and the rear side 232 are connected only at two locations. That is, the front side 230 and the rear side 232 are connected along the edges or transitions therebetween, but are not connected between the edges or transitions. An example of such an arrangement is illustrated in FIG. 9. Such an arrangement can also be referred to herein as a non-interlocked arrangement. In a non-interlocked arrangement, the front side 230 and the rear side 232 are able to roll or slide relative to one another in response to a shear force applied to one or both sides 230, 232.

With reference to FIGS. 5-10, in some configurations, at least a portion of the rear portion 162 has a structure in which the front side 230 and the rear side 232 are joined to inhibit or prevent relative movement between the front side 230 and the rear side 232. As used herein, the front side 230 and the rear side 232 being joined means that movement between the front side 230 and the rear side 232 is restricted or inhibited relative to the tubular arrangement. In some configurations, movement between the front side 230 and the rear side 232 can be prevented or substantially prevented. Thus, in a joined arrangement, the front side 230 and the rear side 232 resist relative sliding movement in response to a shear force applied to one or both sides 230, 232. Such an arrangement can also be referred to as a non-tubular arrangement.

The front side 230 and the rear side 232 can be joined during the knitting process. The front side 230 and the rear side 232 can be joined as a part of the knitting process. In some configurations, the front side 230 and the rear side 232 can be interlocked during the knitting process. Interlocking of the front side 230 and the rear side 232 can involve one or more yarns crossing over from the front side 230 and the rear side 232 during the knitting process. An example of an interlocked arrangement between the front side 230 and the rear side 232 is illustrated in FIG. 10.

The interlocking can be configured to provide a desired amount of resistance to movement between the front side 230 and the rear side 232 in response to a force (e.g., a shear force) applied to one or both of the front side 230 and the rear side 232. In addition, the interlocked portions can allow for easier attachment of connectors (e.g., connector 350 described further below). The interlocked portions can provide reinforcement and structure relative to a non-interlocked or tubular structure, which is beneficial for stability of the interlocked portion of the rear portion 162 or other portion of the headgear 154. For example, additional stability of a connection region between the rear portion 162 and the front portion 160 of the headgear 154 can be beneficial, as this is often an area of high stress. Interlocked portions can hold their shape better than tubular portions to assist in usability of donning and doffing the headgear 154 and the interface assembly 100. Interlocking can also provide increased strength relative to a tubular arrangement. When done in localized areas, such as a strap attachment region (e.g., front strap attachment region), interlocking can provide increased strength in a higher stress area, which allowing another portion or a remainder of the headgear 154 (e.g., the straps 170, 172 and/or portions of the junctions 180) to remain more flexible than the interlocked region(s).

In other configurations, the front side 230 and the rear side 232 can be joined by other arrangement instead of or in addition to interlocking. For example, the front side 230 and the rear side 232 can be joined by stitching, thermoforming, gluing (or other adhesives) or welding. In other arrangements, the front side 230 and the rear side 232 can be joined by overmolding a plastic material onto the front side 230 and the rear side 232. The front side 230 and the rear side 232 could also by joined by introducing a molten plastic material between the front side 230 and the rear side 232 and allowing the molten plastic to cool and solidify.

FIGS. 5-8 illustrate several examples of rear portions 162 having different interlocked and tubular regions. The interlocked regions are illustrated by the patterned portions and the tubular regions are illustrated by the solid portions. As described above, the straps 170, 172 can merge to form the gussets 174. In particular, the front side 230 and the rear side 232 of the straps 170, 172 can merge with one another to form the respective front side 230 and rear side 232 of the gussets 174. Together, the merged straps 170, 172 and gusset 174 can form the junction 180. Accordingly, a thickness of the junction 180 can be equal to or less than a thickness of one or the other (or both) of the first strap 170 or the second strap 172. In some configurations, portions of the junction 180 that are joined may have a smaller thickness than the thickness of one or both of the first strap 170 or the second strap 172. Such an arrangement avoids the substantial increase in thickness (e.g., doubling or close to doubling in thickness) that could occur if a laminated fabric material were used to create a bifurcated headgear structure and two layers of material (e.g., two straps or a strap and a gusset) were overlapped and secured together.

FIG. 5 illustrates an arrangement in which only a portion of the junctions 180 are interlocked. In particular, an outer portion of each of the junctions 180 is interlocked. An inner portion of each of the junctions 180 is tubular. In the illustrated arrangement, an entirety of each of the first strap 170 and the second strap 172 is tubular. However, in other arrangements, a portion or an entirety of the straps 170, 172 could be interlocked.

FIG. 6 illustrates an arrangement in which an entirety of each of the junctions 180 are interlocked. In the illustrated arrangement, an entirety of each of the first strap 170 and the second strap 172 is tubular. However, in other arrangements, a portion or an entirety of the straps 170, 172 could be interlocked.

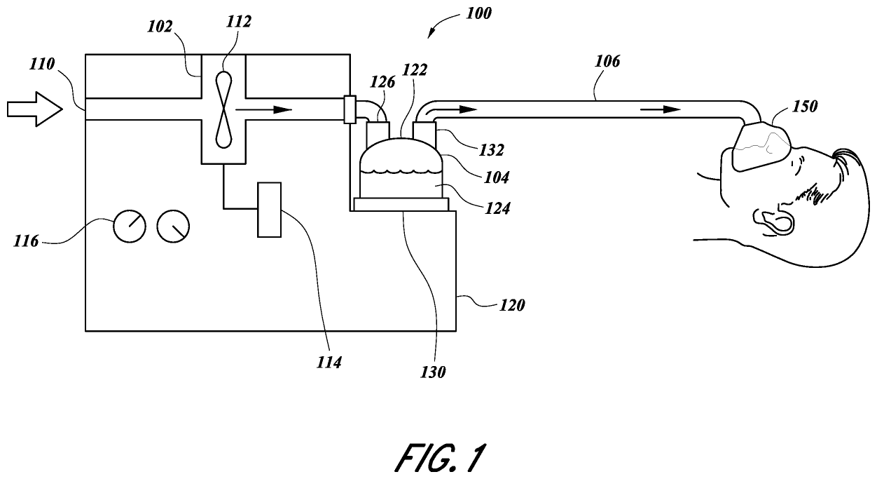

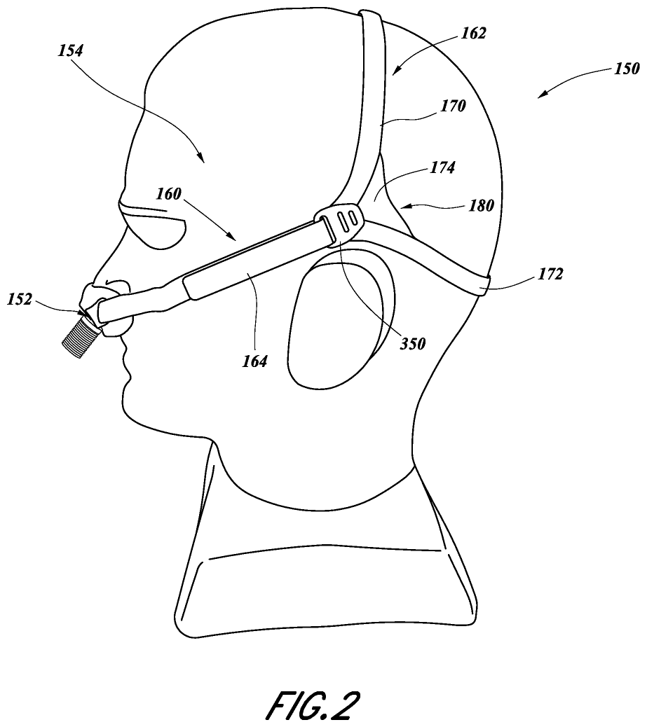

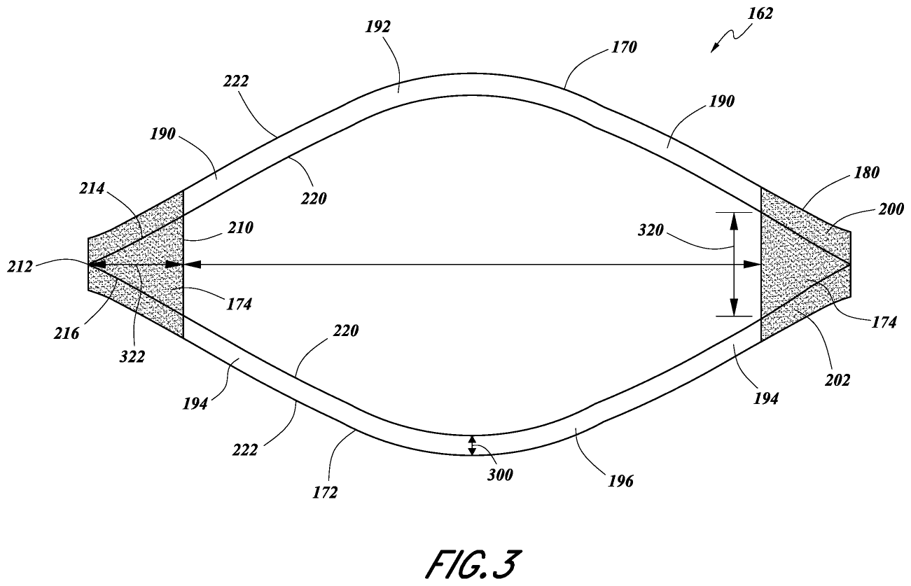

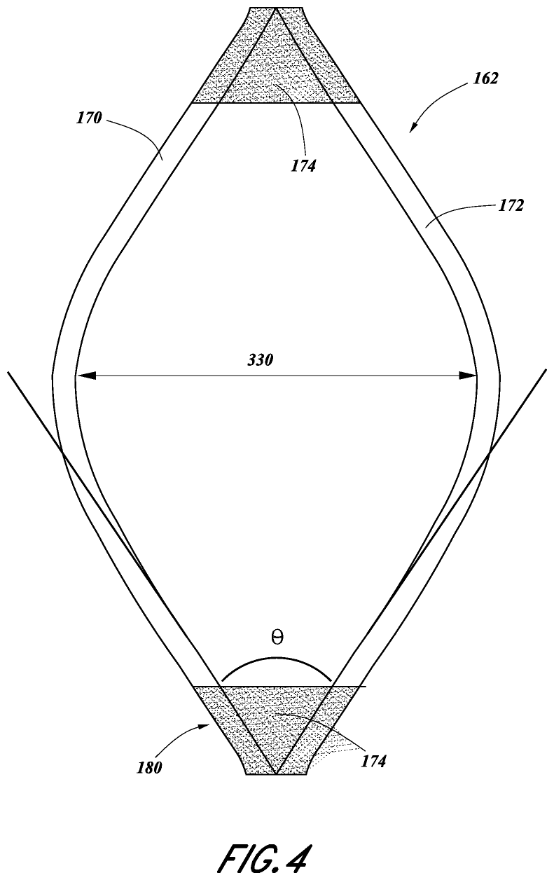

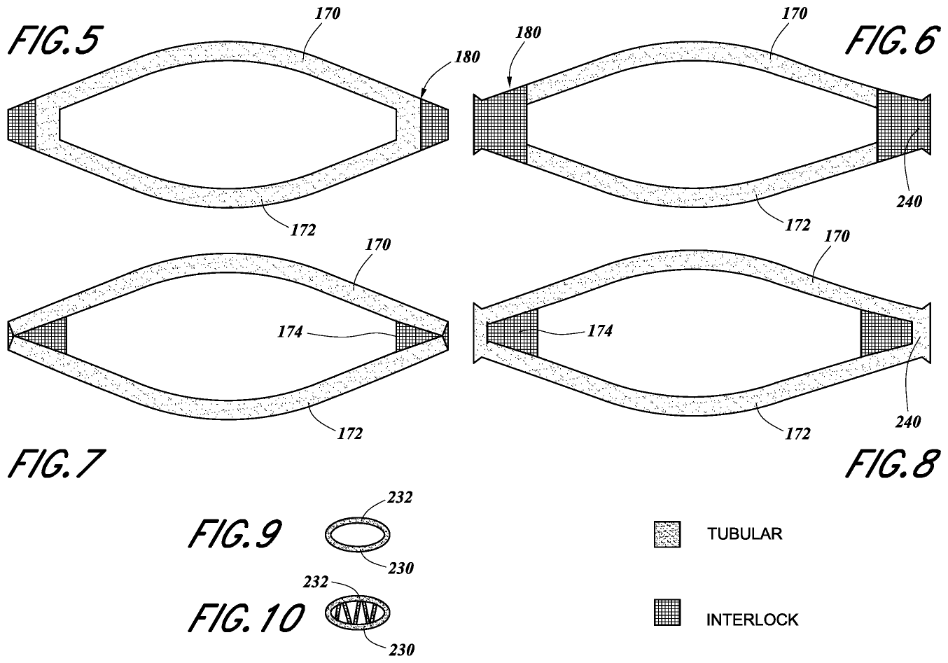

FIGS. 7 and 8 each illustrates an arrangement in which only a portion of the junctions 180 are interlocked. In the arrangement of FIG. 7, the gussets 174 are interlocked. The straps 170, 172 are tubular, with the exception of the tips of the straps 170, 172. In the illustrated arrangement, a triangular portion of the tips of the straps 170, 172 are interlocked. In the arrangement of FIG. 8, gussets 174 are interlocked. The straps 170, 172 are tubular.

In FIGS. 5 and 7, the straps 170, 172 come together such that the outer end 212 of the gussets 174 is a point. In FIGS. 6 and 8, the straps 170, 172 are spaced apart such that the outer end or edge 212 of the gussets 174 is a line having a length. In addition, the arrangements of FIGS. 6 and 8 include a bridge or joining portion 240 that joins the separated ends of the straps 170, 172 in each of the junctions 180. In FIG. 6, the bridge or joining portions 240 are interlocked. In FIG. 8, the bridge or joining portions 240 are tubular.

With particular reference to FIGS. 3 and 4, certain example dimensions or dimensional ranges of the rear portion 162 are described. As shown, the first strap 170 and the second strap 172 each have a strap width 300. In the illustrated arrangement, the strap width 300 of the straps 170, 172 are equal or approximately equal to one another. In other arrangements, the widths 300 of the straps 170, 172 are different from one another. For example, the width 300 of the first strap 170 can be greater than the width of the second strap 172. Alternatively, the width 300 of the second strap 172 can be greater than the width of the first strap 170. In some configurations, the strap width 300 is between about 5-15 mm. In some configurations, the strap width 300 is 10 mm or about 10 mm.

Each of the straps 170, 172 defines a strap length, which can be defined by a line extending between the ends 190, 194 of the straps 170, 172. The strap lengths can be the same as one another or different from one another. For example, the strap length of the first strap 170 can be greater than the strap length of the second strap 172. Alternatively, the strap length of the second strap 172 can be greater than the strap length of the first strap 170. In some configurations, the strap length of the first strap 170 is equal to or substantially equal to the strap length of the second strap 172.

The shape of the strap 170, 172 can impact the strap length, but may not have an equal effect on the overall length of the rear portion 162. Accordingly, the rear portion 162 can be considered to have an effective strap length 310 defined as the distance between the inner edges 210 of the gussets 174. To the extent that the inner edges 210 of the gussets 174 are not parallel to one another, the effective strap length 310 can be defined by an average distance between the edges 210, a minimum distance between the edges 210, a maximum distance between the edges 210, or another particular distance between the edges 210. In some configurations, the effective strap length 310 is between about 150 mm-250 mm, between about 175 mm-225 mm, or between about 190 mm-200 mm, about 200 mm. In some configurations, the effective strap length 310 is 195 mm or about 195 mm.

The inner edges 210 of the gussets 174 can define a gusset width 320. The gusset widths 320 of the gussets 174 can be equal or substantially equal. In other arrangements, gusset widths 320 of the gussets 174 can be different. Similarly, the outer ends or edges 212 of the gussets 174 can define a width that can be the same or different. In some configurations, the outer end or edge 212 of the gusset 174 has a smaller dimension or width than the gusset width 320 of the inner edge 210. For example, the dimension or width of the outer end or edge 212 of the gusset 174 is between one-tenth and one-half, or one-fifth and one-third, or is about one-third of the gusset width 320 of the inner edge 210.

In some configurations, the gusset width 320 of one or both of the gussets 174 is greater than the strap width 300 of one or both of the first and second straps 170, 172. In some configurations, the gusset width 320 of one or both of the gussets 174 is at least twice, at least three times, or at least five times as large as the strap width 300 of one or both of the first and second straps 170, 172. In some configurations, the gusset width 320 can be between about 15 mm-45 mm, between about 20 mm-40 mm, or between about 25 mm-35 mm. In some configurations, the gusset width 320 is about 30 mm.

A distance between the inner edge 210 and the outer end or edge 212 of each of the gussets 174 defines a gusset length 322. The gusset length 322 can be taken in a direction along or parallel with the direction of the effective strap length 310. In some configurations, the gusset length 322 is between about 15 mm-45 mm, between about 20 mm-40 mm, or between about 25 mm-35 mm. In some configurations, the gusset length 322 is 30 mm or about 30 mm.

The upper edge or upper side 214 and the lower edge or lower side 216 of each of the gussets 174 can define a gusset angle θ between them. In some configurations, the gusset angle θ can be between about 30-90 degrees, between about 45-85 degrees, or between about 60-80 degrees. In some configurations, the gusset angle θ is about 70 degrees.

The rear portion 162 can be configured to permit movement of the strap 170, 172 in response to a force applied to the straps 170, 172 tending to separate them. In response to such a force, the gussets 174 can be configured such that the first and second straps 170, 172 can be oriented to form a gusset angle θ between about 30-180 degrees in response to an application of force tending to separate the first and second straps.

The rear portion 162 can be configured to define a maximum distance 330 between the inner edges 220 of the first strap 170 and the second strap 172. The maximum distance 330 can be taken in a direction that is perpendicular or substantially perpendicular to the direction of the effective strap length 310. In some configurations, the maximum distance 330 is between about 20 mm-150 mm, between about 25 mm-100 mm, or between about 30 mm-50 mm. In some configurations, the maximum distance 330 is 38 mm or about 38 mm.

In some configurations, the rear portion 162 is configured such that the effective strap length 310 is between about 250 mm-375 mm, between about 275 mm-350 mm, or between about 300 mm-325 mm when a force of 10 Newtons is applied in a direction aligned with or parallel to the direction of the effective strap length 310. In some configurations, the rear portion 162 is configured such that the effective strap length 310 is 315 mm or about 315 mm when a force of 10 Newtons is applied in a direction aligned with or parallel to the direction of the effective strap length 310.

As described above and shown in FIG. 2, the rear portion 162 is configured for attachment to the front portion 160 of the headgear 154, which can comprise one or more front strap(s) 164. In some configurations, the junctions 180 of the rear portion 162 are configured to be connected to the front portion 160 or the front strap(s) 164. In some configurations, each of the junctions 180 can include a connector 350 that is configured to connect to the front portion 160 or the front strap(s) 164. The illustrated connector 350 is a plastic component that is overmolded to the junction 180 and defines an aperture configured to receive the front strap 164.

A size of an area of overlap between the connector 350 and the junction 180, or an area of the junction 180 covered by the connector 350, can vary. The overlap area can be selected relative to an area of the interlock region of the junction 180 (as illustrated in FIGS. 5-8). In some configurations, the area of the interlock region is greater than the overlap area. In some configurations, the area of the interlock region is less than the overlap area. In some configurations, the area of the interlock region is equal to or substantially equal to the overlap area.

However, other arrangements for connecting the junctions 180 of the rear portion 162 to the front portion 160 or the front strap(s) 164 of the headgear 154 can be used. For example, in some configurations, each of the junctions 180 comprises an open-work structure that defines an aperture or attachment opening configured to receive the front strap 164. Alternatively, an aperture or attachment opening could be created by stitching.

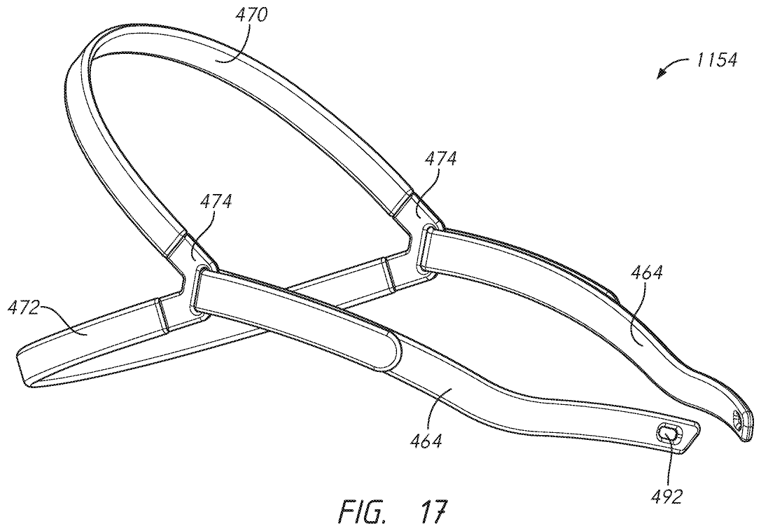

In some configurations, portions or an entirety of the rear portion 162 of the headgear 154 can be provided with a non-flat shape. Shaping of the rear portion 162 can be accomplished by the knitting process or by other processes, such as folding, pleating or stitching. In some configurations, the rear portion 162 is provided with a curved shape by varying the stitch length or loop length between the front side 230 and the rear side 232. For example, constructing the loop length of the front side 230 to be smaller than the loop length of the rear side 232 biases the rear portion 162 into a curved shape with the outer ends being located forward of the central portion so as to better conform to the shape of the user's head, as illustrated by the rear portion 162 as shown in FIG. 14B. In contrast, constructing the loop length of both sides 230, 232 to be equal can provide a flat shape to the rear portion 162, as illustrated by the rear portion 162 as shown in FIG. 14A.

In some configurations, a portion or an entirety of one or both of the first and the second straps 170, 172 has varying loop length between the front side 230 and the rear side 232. For example, the front side 230 has smaller loop length than the rear side 232 to create curvature along a length of the strap. In some configurations, a portion or an entirety of one or both the junctions 180 has varying loop length between the front side 230 and the rear side 232, alone or in addition to varying loop length in one or both of the straps 170, 172. For example, the front side 230 has smaller loop length than the rear side 232 to create curvature along a length of the junction 180.

In some configurations, different types of yarns can be used in the knitting process to create the knitted rear portion 162 or other portions of the headgear 154. For example, as illustrated in FIGS. 15A and 15B, one or more of the straps 170, 172, the gussets 174 or the junctions 180 can include one or more relatively inelastic yarns 400 and one or more relatively elastic yarns 402. Such an arrangement can be created by, for example, a plating process during knitting. In a plating process, one yarn (e.g., 400) forms a base yarn and another yarn (e.g., 402) forms a face yarn. Both yarns 400, 402 are fed together during the knitting process. In some configurations, the relatively inelastic yarn 400 is a nylon yarn (e.g., textured nylon yarn) and the relatively elastic yarn 402 is a nylon (e.g., textured nylon yarn) wrapped elastane or spandex fiber.

An example method of making the rear portion 162 of the headgear 154 is described with reference to FIGS. 11, 12 and 16. With particular reference to FIG. 16, a method 500 of making the rear portion 162 can include knitting a first strap to have a tubular structure at block 502. The method 500 also includes knitting a second strap to have a tubular structure at block 504. The illustrated method 500 includes knitting a junction to couple the first strap to the second strap at block 506. At block 508, a front side and a rear side of at least a portion of the junction are joined.

In some configurations, the knitting of the junction is done prior to the knitting of the first strap and the second strap. In some configurations, the knitting of the junction is done after the knitting of the first strap and the second strap. In some configurations, a first junction is knitted prior to the knitting of the first strap and the second strap and a second junction is knitted after the knitting of the first strap and the second strap. Knitting in this direction can be more efficient than knitting the rear portion in a perpendicular direction at least because the edges requiring binding or having a binding (as described below) are shorter than would be the case when knitting in the perpendicular direction (which would require binding of the outer edges of the straps).

In some configurations, the knitting of at least the first strap and the second strap of blocks 502 and 504 comprises plating or knitting with both a relatively inelastic yarn and a relatively elastic yarn.

In some configurations, the knitting of the junction of block 506 comprises knitting a gusset configured to space apart the first strap portion and the second strap portion.

In some configurations, the knitting of the junction of block 506 comprises binding off an inner edge of the gusset. In some configurations, the knitting of the junction of block 506 comprises binding on an outer edge of the junction. In some configurations, the knitting of the junction of block 506 comprises binding on an inner edge of the gusset. In some configurations, the knitting of the junction of block 506 comprises binding off an outer edge of the junction.

In some configurations, the joining of the front side and the rear side of the junction of block 508 comprises crossing over one or more yarns between the front side and the rear side to interlock the front side and the rear side. In some configurations, the joining of block 508 is done simultaneously with the knitting the junction of block 506. As described above, the interlocking or other manner of joining can be done in a portion or an entirety of the junction. In some configurations, interlocking or another manner of joining can be done in one or both of the straps.

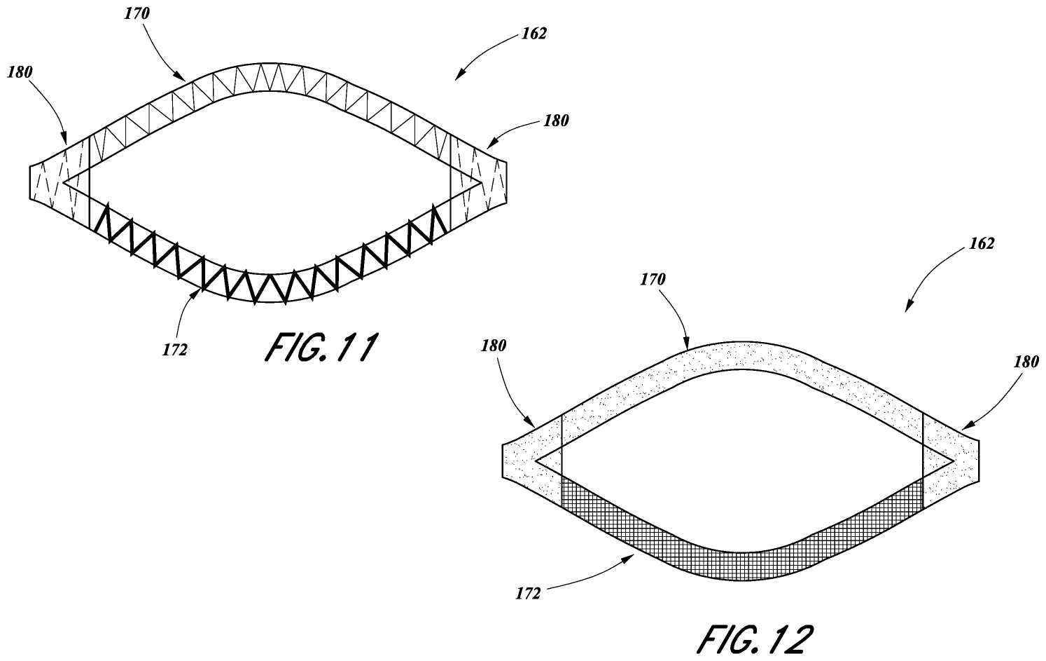

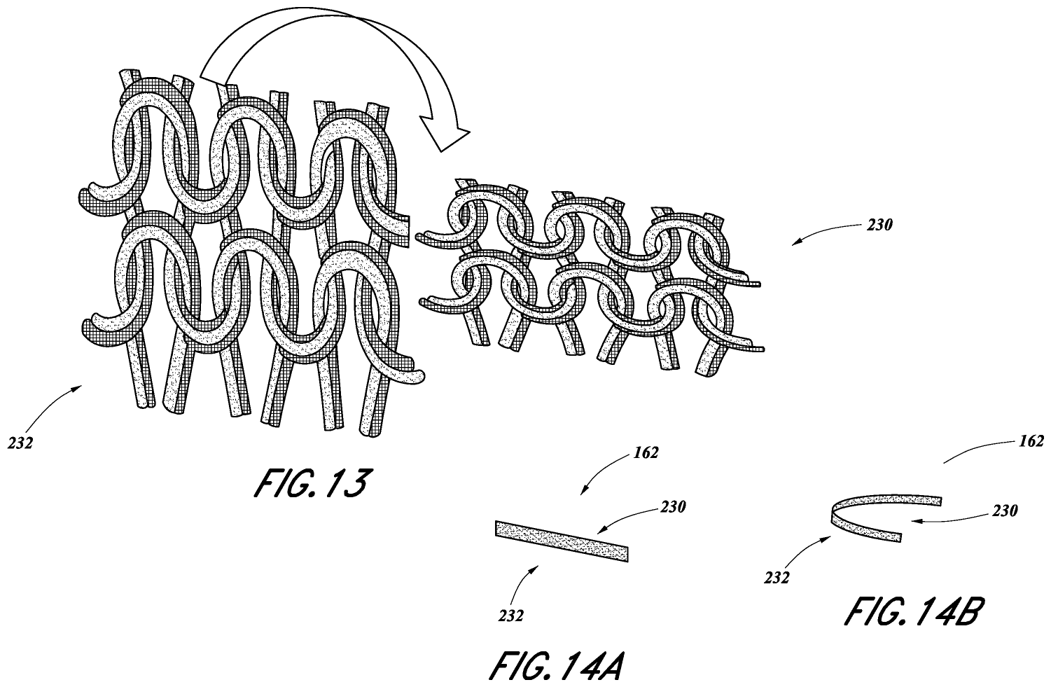

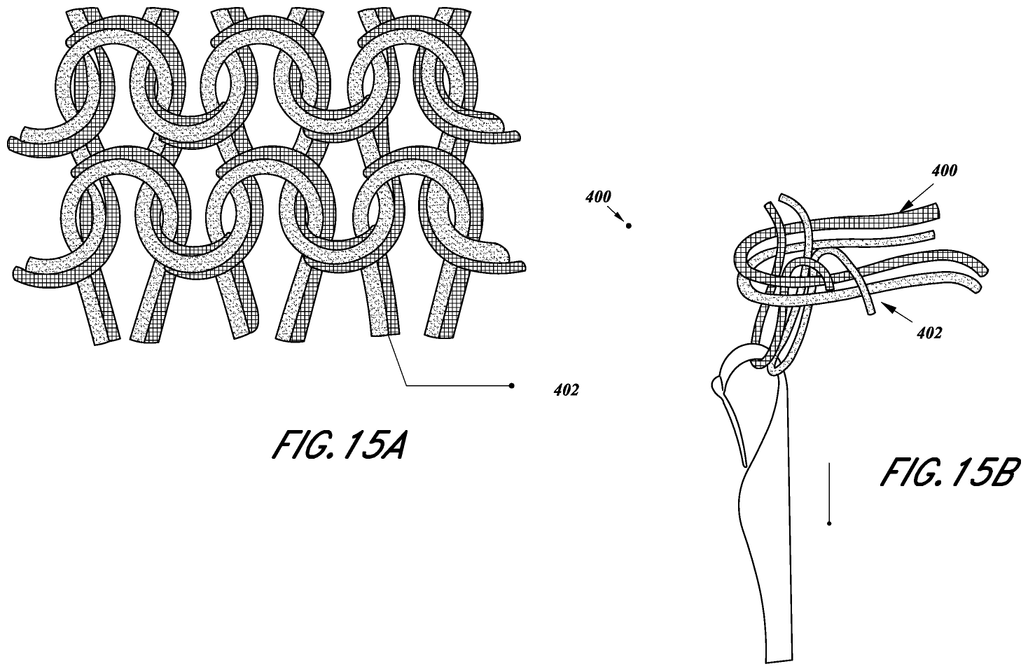

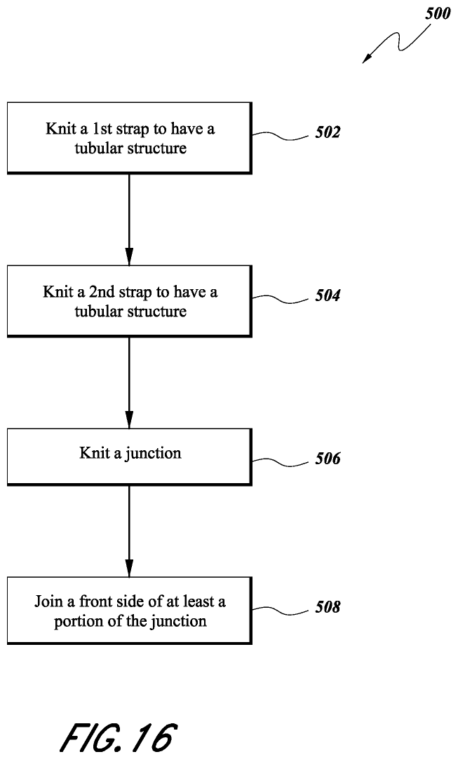

In some configurations, the junction and the first strap are knitted via a first yarn carrier and the second strap is knitted via a second yarn carrier. Such a method is illustrated in FIGS. 11 and 12. FIG. 11 illustrates schematically that the junctions 180 and first strap 170 are knitted via one yarn carrier and the second strap 172 is knitted via a separate yarn carrier or at a separate time than the junctions 180 and first strap 170.

With the direction of knitting of FIGS. 11 and 12 being from the left to the right, the outer edge or end 212 of the first junction 180 involves or requires a bind-on process, the inner edge 210 of the first junction 180 involves or requires a bind-off process, the inner edge 210 of the second junction 180 involves or requires a bind-on process and the outer edge or end 212 of the second junction 180 involves or requires a bind-off process. If the direction of knitting were reversed, the above-described processes would be reversed. As described above, the bind-on and bind-off processes being applied to the ends or edges of the junctions 180 allows for a more efficient knitting process than if the edges 220, 230 of the straps 170, 172 were to require a bind-on and bind-off process due to the much shorter combined length of the edges 220, 230. Binding-off or binding-on can increase the rigidity of the edge, and possibly adjacent regions, of the headgear 154. Therefore, having smaller or minimized edges subject to binding-off or binding-on can be advantageous to reduce or minimize undesired stiffening of the headgear 154. In addition, it can be advantageous to locate the edges subject to binding-off or binding-on in a region where additional stiffness or rigidity is acceptable or even desirable, such as the junctions 180, as opposed to regions where the additional stiffness or rigidity may be undesirable, such as the straps 170, 172. Accordingly, the straps 170, 172 can remain able to flex and stretch for user comfort.

FIG. 17 shows a headgear assembly 1154. The headgear assembly 1154 includes a top strap 470, rear strap 472, and front straps 464. The top strap 470, rear strap 472, and the front straps 464 are connected to brackets 474 located on respective sides of the headgear 1154. In use, the brackets 474 are positioned above the user's left and right ear, respectively.

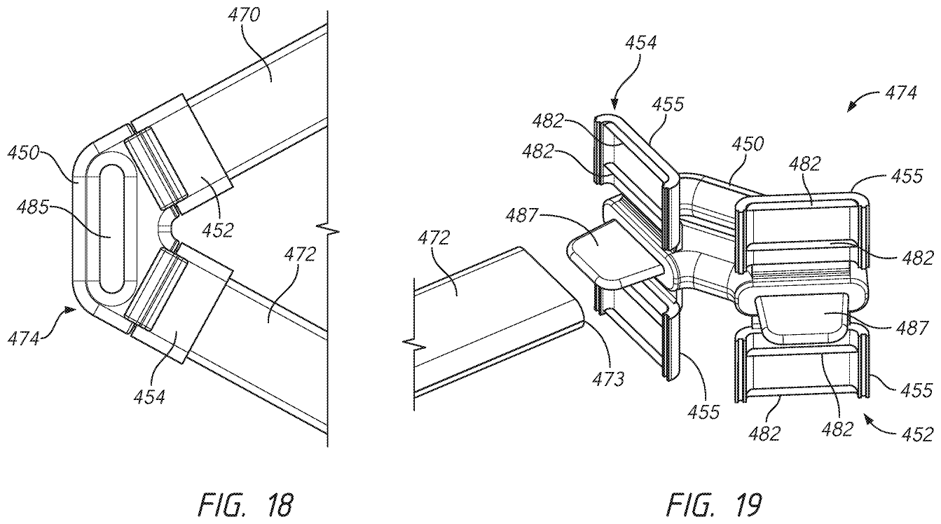

FIG. 18 is a plan view of the bracket 474 as connected to the top strap 470 and rear strap 472. The bracket 474 comprises a front connector 450, an upper connector 452, and a lower connector 454. The front connector 450 includes an elongate slot 485 which is configured to receive one of the front straps 464. The upper connector 452 is connected to the upper strap 470 and the lower connector 454 is connected to the lower strap 472.

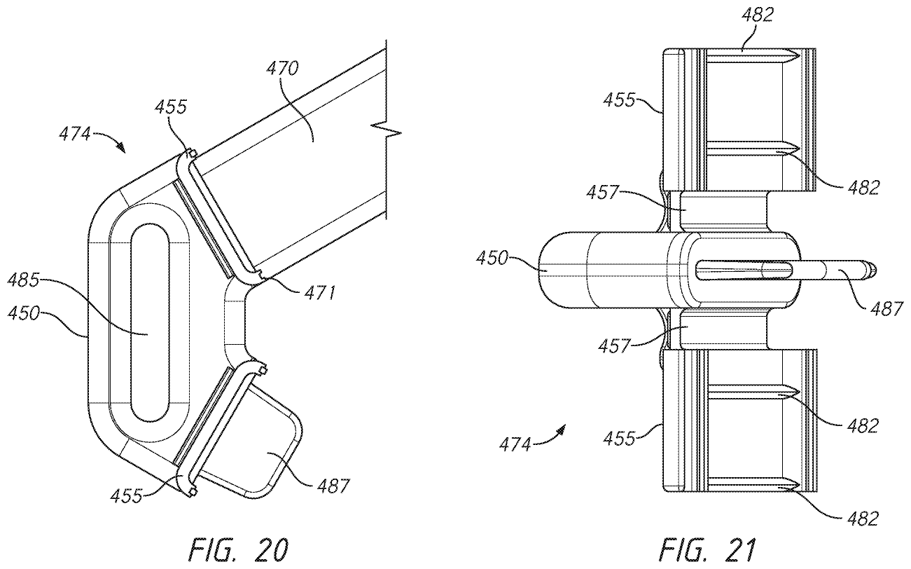

The top strap 470 comprises ends 471 (see e.g., FIG. 20) connected to a respective bracket 474. The top strap 470 extends over the head of the user, between the upper connectors 452 of the brackets 474.

The rear strap 472 comprises ends 473 (see e.g., FIG. 19) that are connected to a respective bracket 474. The rear strap 472 extends across the rear of the user's head, between the lower connectors 454 of the brackets 474.

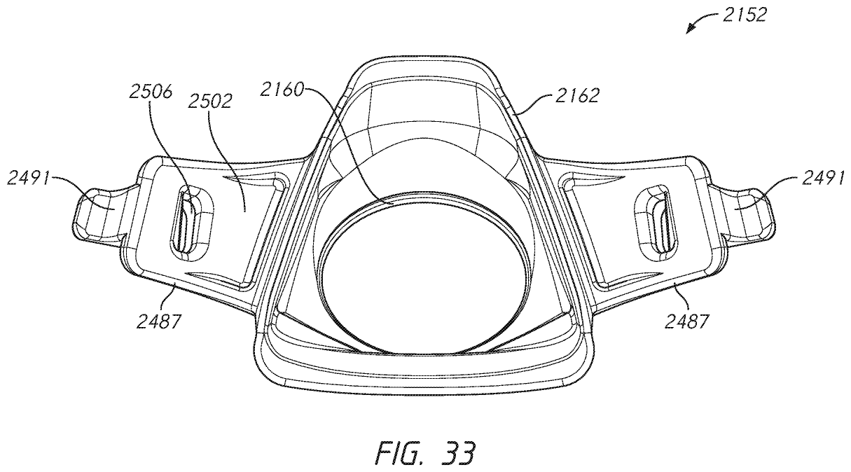

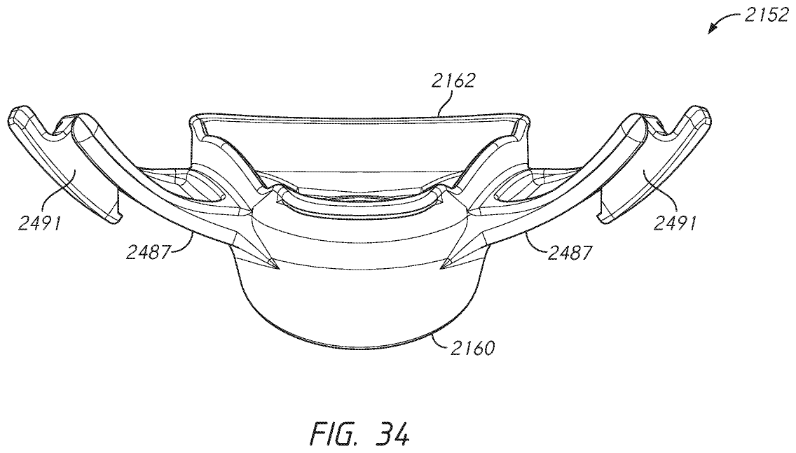

The front straps 464 are configured to extend along the user's cheek and connect the bracket 474 to a mask frame or patient interface (see e.g., FIGS. 33-39). The front connector 450 is connected to the front strap 464. The elongated slot 485 of the front connector 450 receives the front strap 464 and the front strap 464 folds over and attaches to itself in a length adjustable manner, e.g., via a hook and loop or buckle. As shown in FIG. 17, the front straps comprise an opening 492 at the end of the strap 464 configured to connect to the mask frame 2152 (see e.g., FIGS. 33-39).

The front straps 464 are removably connected to the bracket 474, thus different front straps 464 could be provided with the headgear assembly 1154. This would allow for different configurations of front straps 464 to be provided with the headgear assembly 1154. For example, replaceable or alternative front straps 464 could be provided that are configured to connect to different types or styles of mask frames, rigid front straps, padded straps, different materials or colors, or different length straps.

FIGS. 19, 20 and 21 illustrate the bracket 474 in an open position. The upper connector 452 and lower connector 454 comprise an arm 487 that extends from the front connector 450. The arm 487 is inserted into an end 473 of the rear strap 472. Flaps 455 are hingedly connected to the front connector 450 and can be closed to secure the strap 470, 472 to the connector 452, 454.

Ends 471, 473 of straps 470, 472 include a hollow internal space configured to receive a respective arm 487. The straps 470, 472 may comprise a tubular structure, such as a tubular knit strap or a hollow extruded material. Further the straps 470, 472 may include a hollow internal space only at the ends 471, 473 to accommodate the arm 487. In another embodiment, the straps 470, 472 are a laminate material where the ends 471, 473 of the laminate are separated so as to be on either side of the arm 487.

The upper connector 452 comprises an arm 487 that extends from the front connector 450. In the embodiment shown in FIG. 18, the arm 487 is thinner and narrower than the front connector 450. Connected to the front connector 450 is a hinge 457. Adjacent to the hinge 457 is a first flap 455 that is positioned above the arm 487. The first flap 455 is hinged relative to the front connector 450. On the opposite side of the arm 487 is a second hinge 457 and an adjacent flap 455. The hinges 457 are adjacent the elongate slot 485 of the front connector 450. The first flap 455 has an inner surface and outer surface. Energy directors 482 are provided on the inner surface of the first flap 455. The second flap 455 has an inner surface and outer surface. Energy directors 482 are provided on the inner surface of the second flap 455.

The arm 487 of the upper connector 452 is received inside the end 471 of the top strap 470. The hinges 457 allow the flaps 455 to clamp over whichever of the first or second end 471 of the top strap 470 the arm 487 of the upper connector 452 is received in. As such, the flaps 455 are moved towards the arm 487 such that the inner surfaces of the flaps 455 face the surfaces of the arm 487, with a portion of the end 471 of the strap 470 sandwiched between the flap 455 and the arm 487. During a welding operation the material of the energy directors 482 is melted onto the surface of the upper connector 452 of the bracket 474. The melted material creates a permanent connection between the upper connector 452 of the bracket 474 and the outer surface of the upper strap 470.

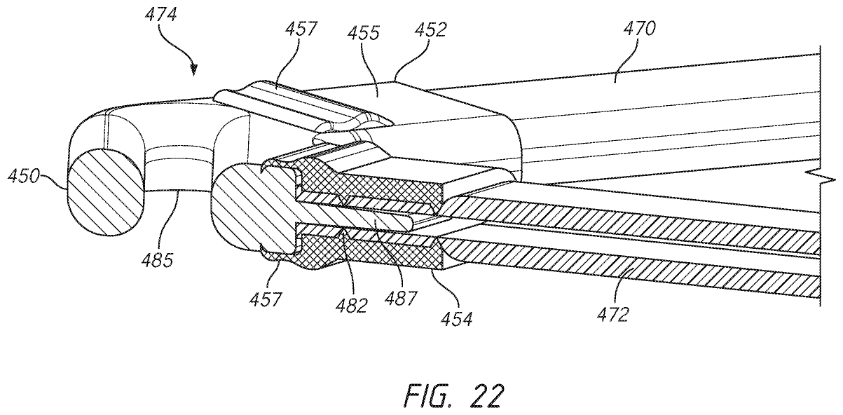

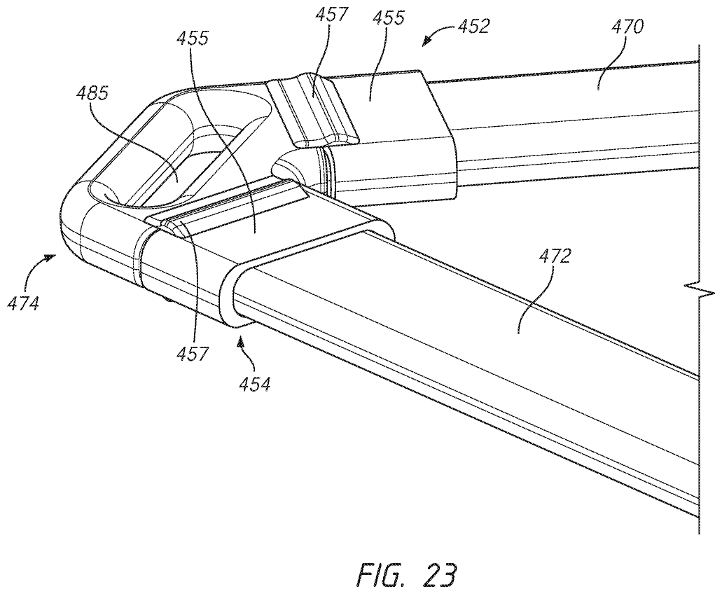

FIGS. 22 and 23 illustrate the bracket 474 in a closed position. In the embodiment in FIG. 22, the rear strap 472 and lower connector 454 are connected by welding. During welding, energy is concentrated toward the energy directors 482 on the flaps 455. The material of the energy directors 482 is melted onto the surface of the respective arm 487, through the sandwich portion of the end 471, 473 of the strap 470, 472. The melted material creates a permanent connection between the lower connector 454 of the bracket 474 and the outer surface of the rear strap 472.

The arms 487 of the upper and lower connectors 452, 454 extend away from the same side of the front connector 450. The arms 487 of the upper and lower connectors 452, 454 extend from the front connector 450 non-parallel to each other. The arms 487 of the upper and lower connectors 452, 454 both extend from the front connector 450 at an angle, α (alpha), to the longitudinal axis of the bracket 474 such that the arms 487 are at an angle 2α to each other.

In the embodiment shown, the lower connector 454 of the bracket 474 is identical to the upper connector 452, such that the bracket 474 is symmetrical about a minor axis of the elongate slot 485. This symmetrical configuration may be advantageous for manufacturing as it can be easier to orient the bracket 474 in a correct manufacturing orientation. In other embodiments, the upper connector 452 and the lower connector 454 may have different configurations, such as different lengths or widths, attachment mechanisms, sizes, angles, or removability.

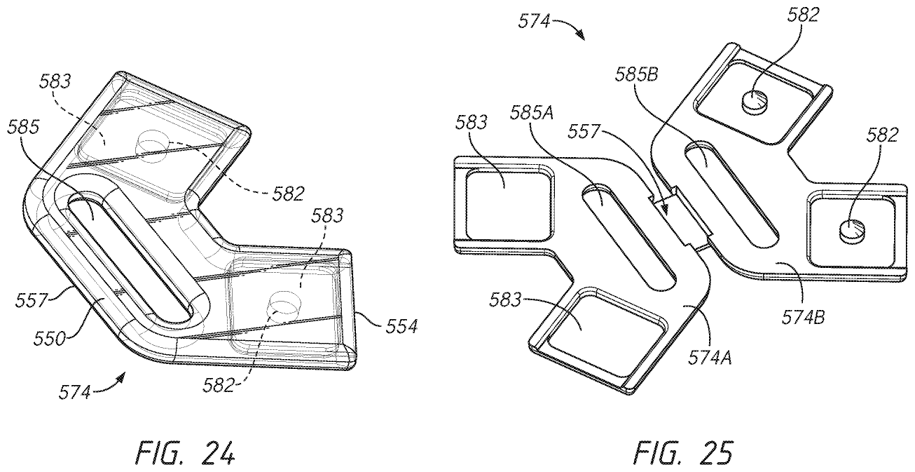

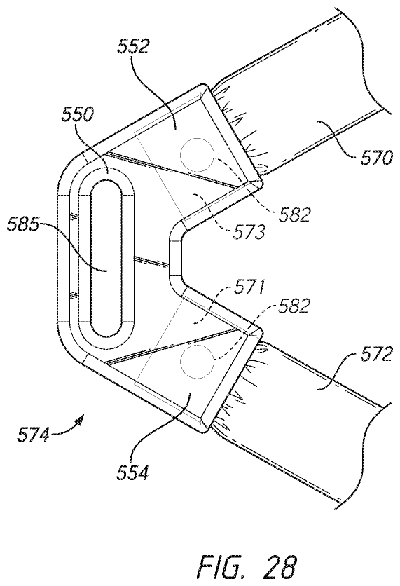

FIGS. 24-29 show a bracket 574 according to another embodiment of the present disclosure. The bracket 574 may be used to join the top strap 470, rear strap 472, and the front straps 464 as part of headgear assembly 1154.

FIG. 24 is a perspective view of the bracket 574 in a closed position. The bracket 574 comprises a front connector 550, an upper connector 552, and a lower connector 554. The front connector 550 includes an elongated slot 585 which is configured to receive the front strap 464. The upper connector 552 is configured to connect to the top strap 470 and the lower connector 554 is connected to the rear strap 472.

FIG. 25 shows the bracket 574 in an open position. The bracket 574 comprises a first bracket portion 574A and a second bracket portion 574B. A hinge 557 connects the first bracket portion 574A to the second bracket portion 574B. The hinge is connected to a periphery of the first bracket portion 574A and the second bracket portion 574B, respectively. Each of the first bracket portion 574A and the second bracket portion 574B comprise an inner surface and an outer surface. Recesses 583 are provided on the inner surface of the first bracket portion 574A. Energy directors 582 are provided on the second bracket portion 574B. In at least some embodiments, the first bracket portion 574A is a mirror of the second bracket portion 574B.

The first bracket portion 574A comprises a first elongate slot portion 585A and the second bracket portion comprises a second elongate slot portion 585B. The hinge 557 is adjacent both the first elongate slot portion 585A and the second elongate slot portion 585B. In at least some embodiments, the first bracket portion 587A, the second bracket portion 587B, and the hinge 557 are integrally formed or integrally moulded.

The hinge 557 allows the first bracket portion 574A to be folded onto the second bracket portion 574B to change from the open position shown in FIG. 25 and change the bracket 574 to a closed position. In the closed position, the inner surfaces of the first bracket portion 574A and the second bracket portion 574B face each other and the recesses 583 form a pocket 586. The elongate slot 585 is formed by elongate slot portions 585A, 585B when the bracket 574 changes to a closed position. The axis of the hinge 557 runs parallel to the longitudinal axis of the elongate slot 585.

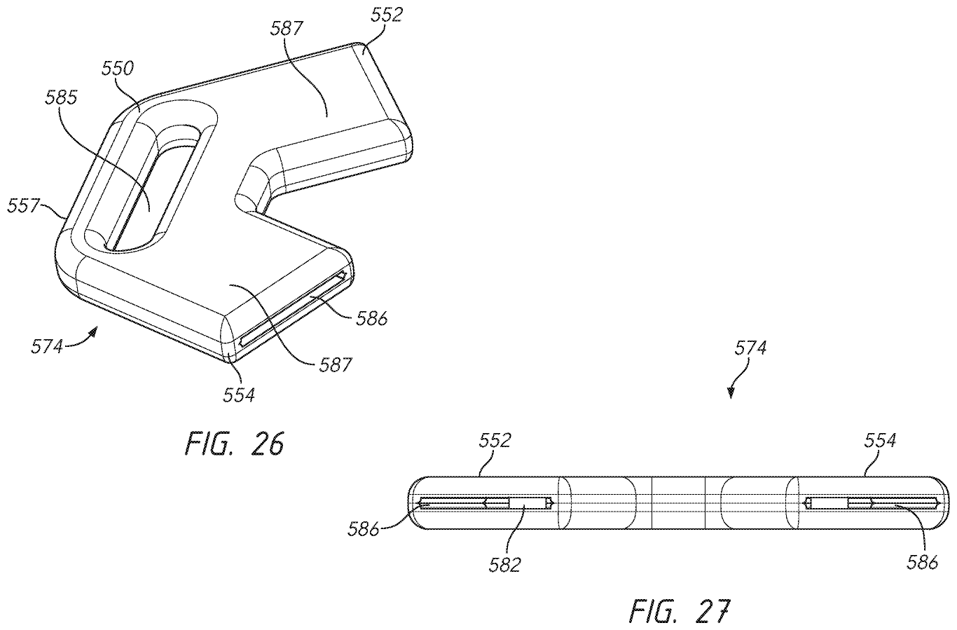

FIGS. 26, 27, and 28 illustrate the bracket 574 in a closed position.

The front connector 550 is connected to, for example, a front strap (e.g., front strap 464). The elongated slot 585 of the front connector 550 receives one of the front straps. The front strap folds over and attaches to itself in a hook and loop fashion. In at least some embodiments, the bracket 574 is symmetrical about a minor axis of the elongate slot 585.

One end 571 of the top strap 570 and one end 573 of the rear strap 572 are positioned onto the recesses 583 of the bracket 574. The hinge 557 allows the first bracket portion 574A to fold on top of the second bracket portion 574B and clamp over one end 571 of the top strap 570 and one end 573 of the rear strap 572 until the first bracket portion 574A is in a closed position. The strap ends 571, 573 are sandwiched between the first bracket portion 574A and the second bracket portion 574B.

In this embodiment, the top strap 570 and upper connector 552 are connected by welding. During welding, energy is concentrated toward the energy director 582 on the first bracket portion 574A. The material of the energy director 582 is melted onto the surface of the upper connector 552. The melted material creates a permanent connection between the upper connector 552 of the bracket 574 and the outer surface of the top strap 570. In this embodiment, the connection between the lower connector 554 and the rear strap 572 is identical to the connection between the upper connector 552 and the top strap 570. An advantage of this connection between the connectors and the straps is that the top strap 570 and the rear strap 572 can be secured to the bracket in a single closing operation.

The upper and lower connectors 552, 554 both extend from the body at an angle, α (alpha) to the longitudinal axis of the bracket 574 such that the arms are at an angle 2α to each other.

In the embodiment shown, the lower connector 554 of the bracket 574 is identical to the upper connector 552, such that the bracket 574 is symmetrical about a minor axis of the elongate slot 585. This symmetrical configuration may be advantageous for manufacturing as it can be easier to orient the bracket 574 in a correct manufacturing orientation. In other embodiments, the upper connector 552 and the lower connector 554 may have different configurations, such as different lengths or widths, attachment mechanisms, sizes, angles, or removability.

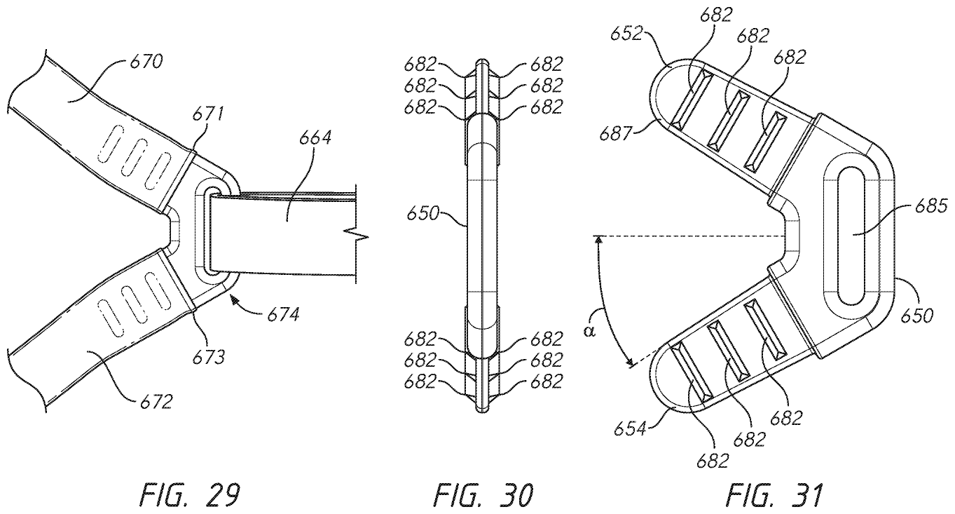

FIGS. 29, 30, and 31, show a bracket 674 according to another embodiment of the present disclosure. The bracket 674 may be used to join the top strap 470, rear strap 472, and the front straps 464 as part of headgear assembly 1154.

FIG. 29 is a plan view of a portion of a headgear assembly. An upper strap 670 and a rear strap 672 are joined to the bracket 674. A front strap 664 is connected to the bracket 674.

FIGS. 30-31 provide additional views of the bracket 674. The bracket 674 comprises a front connector 650, an upper connector 652, and a lower connector 654. The front connector 650 includes an elongated slot 685. The upper connector 652 comprises an arm 687 that extends from the front connector 650. The lower connector 654 comprises an arm 687 that extends from the front connector 650.

The front connector 650 is connected to a front strap 664. The elongated slot 685 of the front connector 650 receives the front strap 664 and the front strap 664 folds over and attaches to itself in a length adjustable manner, e.g., via a hook and loop or buckle.

The arms 687 of the upper and lower connectors 652, 654 extend away from the front connector 650. The arms 687 of the upper and lower connectors 652, 654 extend from the front connector 650 non-parallel to each other. The arms 687 of the upper and lower connectors 652, 654 both extend from the front connector 650 at an angle, α (alpha) to the longitudinal axis of the bracket 674 such that the arms are at an angle 2α (alpha) to each other. The arms 687 are thinner and narrower than the front connector 650 of the bracket 674.

In the embodiment shown, the lower connector 654 of the bracket 674 is identical to the upper connector 652, such that the bracket 674 is symmetrical about a minor axis of the elongate slot 685. This symmetrical configuration may be advantageous for manufacturing as it can be easier to orient the bracket 674 in a correct manufacturing orientation. In other embodiments, the upper connector 652 and the lower connector 654 may have different configurations, such as different lengths or widths, attachment mechanisms, sizes, angles, or removability.

In the embodiment shown in FIG. 29, ends 671, 673 of straps 670, 672 include a hollow internal space configured to receive a respective arm 687. Energy directors 682 are provided on the surface of the arm 687 for securing the arm 687 to the respective strap 670, 672. The energy directors 682 help to concentrate energy during welding, e.g., ultrasonic or RF welding. During welding, the material of the energy directors 682 melts and helps to adhere the strap 670, 672 to the arm 687. The melted material creates a permanent connection between the upper connector 652 of the bracket 674 and the inner surface of the top strap 670.

In the embodiment shown in FIG. 29, ends 671, 673 of straps 670, 672 include a hollow internal space configured to receive a respective arm 687. The straps 670, 672 may comprise a tubular structure, such as a tubular knit strap or a hollow extruded material. Further the straps 670, 672 may only include a hollow internal space only at the ends 671, 673 so as to accommodate the arm 687. In another embodiment, the straps 670, 672 are a laminate material where the ends 671, 673 of the laminate are separated so as to be on either side of the arm 687.

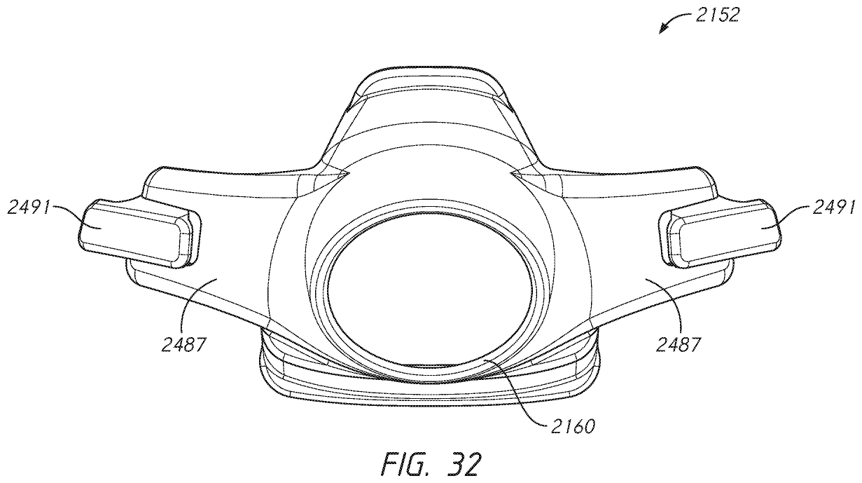

FIGS. 32, 33, 34, and 35 show a mask frame 2155 in the front, rear, top and bottom views, respectively. The mask frame may be used, for example, with embodiments of headgear 1154 discussed above.

The mask frame 2152 includes a mask frame inlet 2160 and a mask frame outlet 2162. In at least some embodiments, the mask frame outlet 2162 also provides a connection for a patient interface for providing gases to a user, e.g., cannula, nasal pillows, nasal, nasal cradle, or full-face masks (not shown). The mask frame 2152 comprises lateral frame portions in the form of side arms 2487. As shown, each side arm 2487 includes a headgear connector 2491 that protrudes from a front surface of the respective side arm 2487. The headgear connector 2491 may be, for example, configured to engage the strap opening 492 of the headgear assembly 1154 described above.

The headgear connector 2491 comprises a post 2493. The headgear connector 2491 engages with a front strap 2764, facilitating connection between the mask frame 2155 and the headgear 2154. The post 2493 provides a mounting location for headgear straps such that the front straps are secured to the mask frame such that they can hold a tension to counteract blow-off force during CPAP therapy.

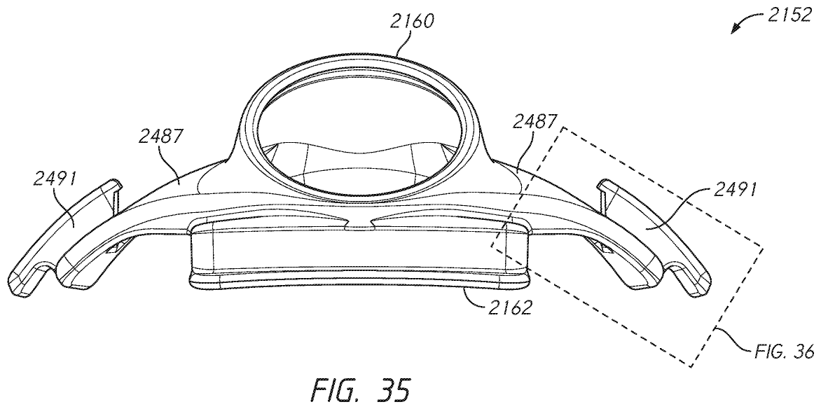

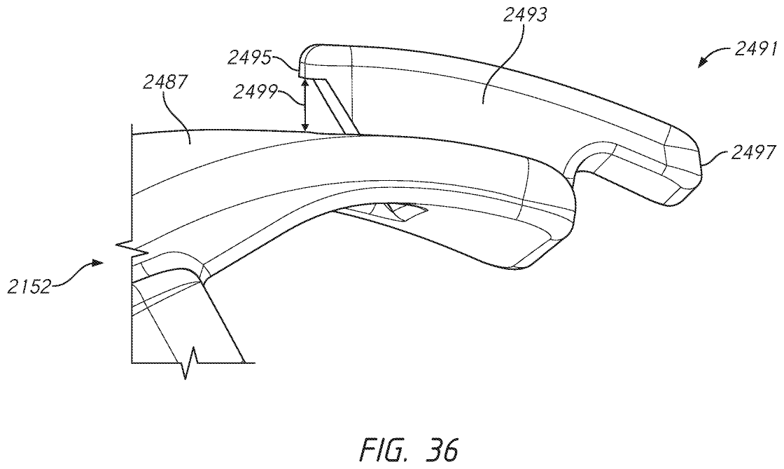

FIGS. 36, 37, and 38 provide additional views of the mask frame. In particular, FIG. 36 shows an enlarged view of the side arm of the mask frame from the bottom view of FIG. 35. As illustrated, the headgear connector 2491 comprises a post 2493 extending from the front surface of the side arm 2487. Extending medially from the post 2493, towards the mask frame inlet 2160, is a medial flange 2495. Between the medial flange 2495 and the front surface of the side arm is a gap 2499. Extending laterally from the post 2493, away from the mask frame inlet 2160, is a lateral flange 2497. In order to form the gap 2499, the medial flange 2495 extends above the opening 2506 that is defined through the lateral frame portion.

In at least some embodiments the post 2493 has a rectangular shape, with a horizontal length greater than its width. The width of the side arm 2487 is greater than the width of the post 2493. The rectangular shape of the post 2493 acts to limit, or at least reduce, the upwards or downwards rotation (i.e. rotation axis normal to front surface of frame arm) between the strap and the frame. The rectangular shape also acts as an indicator of the correct orientation of the strap and frame for assembly.

In the current embodiment, for example as shown in FIG. 36, the lateral flange 2497 has a greater length than the medial flange 2495. The medial flange 2495 secures medial side of the front end of the front strap 2764 while the lateral flange 2497 secures the rear side of the front strap 2764. The flanges reduce accidental removal of the front strap from the post 2493 by providing a physical barrier to the lifting of the strap at the respective sides.

The front surface of the post 2493 (i.e. when mask frame is in the worn orientation) is continuous with the front surfaces of both flanges, producing a flush surface. This reduces the of corners or sharp edges that may cause discomfort or difficulty of cleaning.

The medial flange 2495 has a length of 0.5 mm and on the lateral flange 2497 has a length of 4.2 mm. The medial flange 2495 has a thickness of 1 mm, and the lateral flange 2497 has a thickness of 1.5 mm.

In the worn orientation, the upper and lower walls of the medial and lateral flanges 2495, 2497 are continuous with and are on the same respective planes of the upper and lower walls of the post. In other words, the widths of both medial and lateral flanges 2495, 2497 are equal to that of the post 2493. This forms flat upper and lower surfaces spanning the length of the headgear connector. The absence of an undercut allows the post 2493 and flanges 2495, 2497 to be moulded in a two-part mould tool.

In the non-limiting, exemplary embodiments shown, the gap 2499 between the bottom surface of the medial flange 2495 is approximately 1.6 mm. The lateral flange 2497 extends beyond the lateral edge of the side arm 2487. In use, the medial and lateral flanges 2495, 2497 overlay respective sides of the opening 492 of the front strap 2764.

In at least some embodiments, lateral wall of the post 2493 is continuous with the lateral edge of the side arm 2487. This results in no undercut being present, hence allows the headgear connector 2491 and side arm 2487 to be moulded together, as a single component in a two-part mould tool.

The flanges 2495 and 2497 help to reduce the likelihood of the strap slipping off and disconnecting from the post 2493. Once attached, the opening 492 surrounds post, the thickness of the strap fitting within the gap 2499.

During use, tension is applied to the headgear and mask frame. This tension can become somewhat concentrated on the post and the strap opening. This can cause the strap opening to elongate during use. As such it may be beneficial for the lateral flange to be longer than the medial flange. Additionally, in at least some embodiments, the lateral flange extends beyond a lateral edge of the side arm.

For example, if the rear end of the strap is pulled forward, the flange acts as a physical barrier to the free end of the strap lifting from the frame, therefore helps reduce the chance of accidental removal.

The lateral flange 2497 has a greater strength than the medial flange 2495 due to its greater thickness. This reduces the likelihood of the lateral flange 2497 flexing and potentially breaking with wear or incorrect use. This is useful especially for the lateral flange as it extends beyond the edge of the side arm 2487 and is more exposed to forces and stresses during use.

The lateral flange 2497 follows the general curvature of the side arm 2487, which is shaped to follow the curvature of the user's face. This directs the rear end of the strap inwards towards the user's face to promote a secure fit of the headgear and mask.

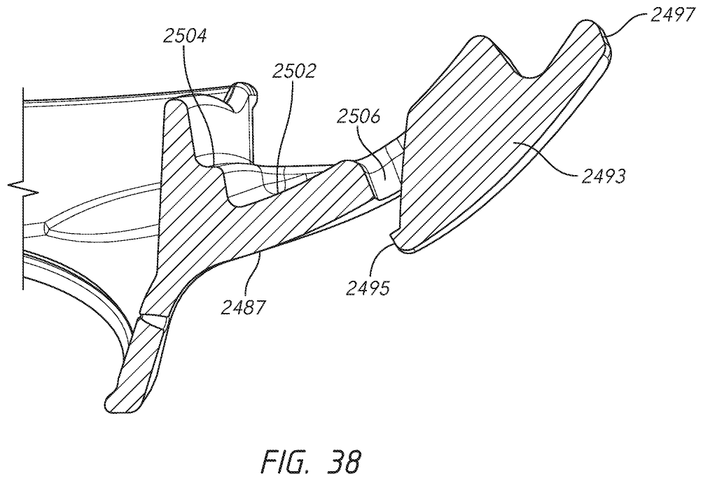

FIG. 38 is a cross-sectional view of the side arm. As illustrated, each side arm 2487 further features an opening 2506 adjacent the medial side of the headgear connector 2491, the opening extending through the entire thickness of the side arm 2487. The opening 2506 extends through the entire thickness of the side arm 2487, therefore allowing the moulding and removal of the frame component in a two-part mould tool. Specifically, the location of the opening allows the medial flange 2495 to be formed in a two-part mould tool.

The underside of the medial flange 2495 can be formed by the same part of the two-part mould tool as the rear side of the frame. In addition, the medial wall of the post 2493 is formed at an incline relative to the front surface of the side arm 2487, forming an acute angle between the medial wall and the front surface of the side arm 2487. This feature further contributes to the ability of the frame component to be moulded and removed from a two-part mould tool.

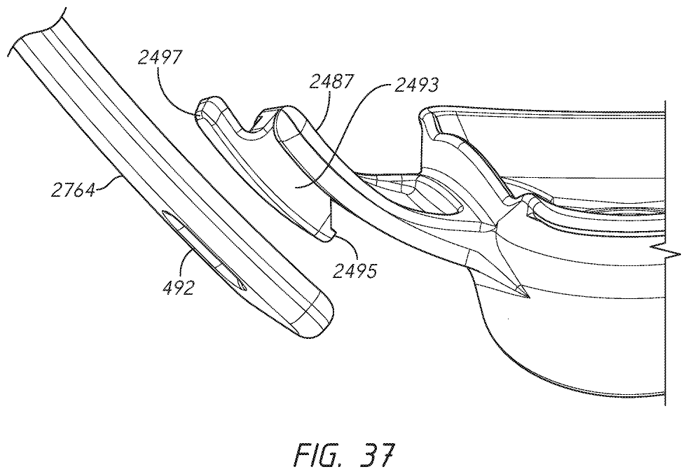

FIG. 37 shows a front strap 2764 and mask frame 2152, with the front strap opening aligned with the headgear connector of the side arm. As illustrated, the front end of each of the front strap 2764 features an opening 492 with a rectangular shape with its long edge parallel to the length of the strap. The opening fits around the post 2493. The distance between the distal edges of the medial and lateral flanges 2495, 2497 is greater than the length of the opening 492, leading to interference between the edges of the opening and the flanges when the opening 492 is connected to the headgear connector 2491. Exemplary dimensions of the length of the strap opening 492 is 6.5 mm, and of the width is 3.9 mm. The thickness of the strap is around 1.6 mm, equal to gap 2499 of the side arm. These dimensions allow the strap and frame to be connected securely.

In an alternative embodiment, the opening may alternatively have an ovular shape or be formed as a slit in the strap. In another alternative embodiment, the thickness of the strap is greater than 1.6 mm and the strap is made of a compressible material.

The strap 2764 is formed of a flexible material such as fabric or silicone. This allows the opening to expand in area, and/or width or length and stretch over the flanges. The opening of the strap is formed through removal of material such as die-cutting or RF crease-cutting. Additional embodiments include a strap featuring a free end overmoulded with TPU or any other resilient material to increase durability, stiffness and usability, the more durable TPU portion with an opening, or a buckle component attached to the strap.

For assembly, the free end of the strap 2764 featuring an opening is pushed onto the headgear connector 2491, and the opening 492 stretches around and over the flanges 2495, 2497 to fit around the post 2493. As the lateral flange is longer than the medial end, the lateral side of the opening 492 is more easily applied first, allowing the medial side to then be stretched over with ease. Once stretched over, connection between the headgear and frame is achieved and the flanges secure the headgear during use.

Each side arm 2487 further includes a recess 2502 on the rear surface. The recess 2502 produces a more consistent thickness of the frame arm along its length. Large thickness variations may produce manufacturing artefacts such as sinks when moulded, specifically in the front surface of the side arm 2487. The abutment located at the medial portion of the rear surface of the frame arm is the location at which the frame interacts with the seal. The frame supports the seal, therefore, a greater thickness relative to the frame arms is advantageous at this location. Thus, the recess 2502 extends from a location adjacent the abutment along the length of the frame arm.

### CONCLUSION

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

Moreover, the following terminology may have been used herein. The singular forms “a,” “an,” and “the” include plural referents unless the context clearly dictates otherwise. Thus, for example, reference to an item includes reference to one or more items. The term “ones” refers to one, two, or more, and generally applies to the selection of some or all of a quantity. The term “plurality” refers to two or more of an item. The term “about” or “approximately” means that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. The term “substantially” means that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “about 1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “about 1 to about 3,” “about 2 to about 4” and “about 3 to about 5,” “1 to 3,” “2 to 4,” “3 to 5,” etc. This same principle applies to ranges reciting only one numerical value (e.g., “greater than about 1”) and should apply regardless of the breadth of the range or the characteristics being described. A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

1.-57. (canceled)

58. A mask frame, comprising:
a mask frame inlet and a mask frame outlet;
at least one lateral frame portion comprising a front surface and a rear surface;
a headgear connector extending from the front surface; and
an opening defined through the at least one lateral frame portion, from the front surface to the rear surface, and positioned between the headgear connector and the mask frame inlet.

59. The mask frame according to claim 58, wherein the headgear connector comprises a post positioned adjacent to the opening.

60. The mask frame according to claim 59, further comprising a medial flange extending medially from the post.

61. The mask frame according to claim 60, wherein a medial flange extends above the opening.

62. The mask frame according to claim 60, further comprising a gap between the medial flange and the front surface of the at least one lateral frame portion.

63. The mask frame according to claim 60, further comprising a lateral flange extending laterally from the post.

64. The mask frame according to claim 60, wherein a width of the at least one lateral frame portion is greater than a width of the post.

65. The mask frame according to claim 64, the width of the post is substantially the same as a width of the medial flange.

66. The mask frame according to claim 63, a width of the post is substantially the same as a width of the lateral flange.

67. The mask frame according to claim 63, wherein a length of the lateral flange is greater than a length of the medial flange.

68. The mask frame according to claim 67, wherein the length of the lateral flange is approximately 4.2 mm and the length of the medial flange is approximately 0.5 mm.

69. The mask frame according to claim 63, wherein a thickness of the lateral flange is greater than a thickness of the medial flange.

70. The mask frame according to claim 69, wherein the thickness of the lateral flange is approximately 1.5 mm and the thickness of the medial flange is approximately 1 mm.

71. The mask frame according to claim 63, wherein the lateral flange extends beyond a lateral edge of a side arm.

72. The mask frame according to claim 63, wherein a front surface of the post is continuous with a front surface of the medial flange and a front surface of the lateral flange.

73. The mask frame according to claim 72, wherein the front surface of the lateral flange has a curvature following the curvature of a user's face.

74. The mask frame according to claim 59, wherein the post has a rectangular shape or cross-section.

75. The mask frame according to claim 59, wherein the post has a length of 6.5 mm and a width of 3.9 mm.

76. The mask frame according to claim 58, further comprising a recess positioned between the opening and the mask frame inlet.

77. The mask frame according to claim 76, wherein the recess is positioned on a rear surface of a side arm.

78. A headgear strap configured to mate with the headgear connector according to claim 58, wherein the headgear strap has an opening configured to secure to the headgear connector.

79. The headgear strap according to claim 78, wherein the opening is located on a free end of the headgear strap.

80. The headgear strap according to claim 78, wherein the opening has a rectangular shape corresponding with a shape of a post.

81. The headgear strap according to claim 78, wherein, when assembled, a thickness of the headgear strap fits within a gap.

82. The headgear strap according to claim 78, wherein, when assembled, a medial flange and a lateral flange hang over the headgear strap.

83. The headgear strap according to claim 78, wherein the headgear strap is formed of a flexible material.

84. The headgear strap according to claim 78, wherein the opening is formed through removal of material such as die-cutting and RF crease-cutting.

85. The headgear strap according to claim 79, wherein the free end of the headgear strap comprises an overmoulded section formed with a resilient material.
